(19) United States
(12) Patent Application Publication
(10) Pub. No.: US 2025/0179031 A1
(43) Pub. Date: Jun. 5, 2025

(54) PROTEASOME ENHANCERS AND USES THEREOF

(71) Applicant: Board of Trustees of Michigan State University, East Lansing, MI (US)

(72) Inventors: Robert A. Mosey, Sault Sainte Marie, MI (US); Jetze J Tepe, East Lansing, MI (US)

(21) Appl. No.: 18/833,698

(22) PCT Filed: Jan. 27, 2023
(86) PCT No.: PCT/US2023/061470

§ 371 Date: Jul. 26, 2024

Related U.S. Application Data
(60) Provisional application No. 63/305,862, filed on Feb. 2, 2022.
(60) Provisional application No. 63/303,986, filed on Jan. 28, 2022.

(51) Int. Cl. C07D 239/74; A61K 31/517; A61K 31/519; A61K 31/55; C07D 401/04; C07D 405/06; C07D 471/04; C07D 471/18; C07D 487/04

(52) U.S. Cl. CPC C07D 239/74; A61K 31/517; A61K 31/519; A61K 31/55; C07D 401/04; C07D 405/06; C07D 471/04; C07D 471/18; C07D 487/04

(57) ABSTRACT

Described herein are compounds, methods for making such compounds, and the use of such compounds in the treatment of cancer, an inflammatory disease or condition or neurodegenerative diseases, such as Parkinson's disease, Alzheimer's disease, Huntington's disease, and ALS.

### CROSS-REFERENCE TO RELATED APPLICATIONS

This application claims the benefit of U.S. Provisional Appl. Nos. 63/303,986, filed Jan. 28, 2022; and 63/305,862 filed Feb. 2, 2022 both of which is incorporated by reference as if fully set forth herein.

### STATEMENT OF GOVERNMENT SUPPORT

This invention was made with government support under 1 R01 AG066223-01A1 and T32GM092715 awarded by the National Institutes of Health and MRI Award 1626523 awarded by the National Science Foundation. The government has certain rights in the invention.

### BACKGROUND

The regulation of protein synthesis, degradation, folding, trafficking and aggregation within a cell are collectively known as proteostasis. Proteostasis is maintained by a wide array of cellular machinery that work to ensure that proteins are present in the proper location, amounts and form to perform their respective functions. When one of the pathways involved with proteostasis becomes dysregulated there can be disastrous effects on the cell and even on neighboring cells. One increasingly prevalent example of this is seen in neurodegenerative diseases, such as Parkinson's disease (PD), Alzheimer's disease (AD), Huntington's disease (HD) and amyotrophic lateral sclerosis (ALS). In these neurodegenerative diseases, accumulation of specific aggregation-prone proteins (hereafter referred to as intrinsicaly disordered proteins (IDPs)) leads to toxic signaling and disruption of proteostasis caused by their uncontrolled aggregation and oligomerization (hereafter, aggregation and oligomerization are used interchangeably). For example, the IDP α-synuclein (α-syn) and its oligomers are associated with the pathogenesis of PD. IDPs are named for their lack of tertiary structure allowing them to adopt numerous conformations and interact with multiple binding partners. IDPs are generally short-lived signaling proteins or transcription factors that are highly bound to other cellular components keeping free cytosolic levels low. Additionally, unbound IDPs are readily degraded by the 20S proteasome, the default protease responsible for IDP digestion. The accumulation of IDPs seen in neurodegenerative diseases can begin as a result of one of several disruptions (e.g. mutations, changes in expression, oxidative stress, aging, proteasome impairment, etc.) to their normal regulation. While α-syn may not be the sole cause of PD, there is strong evidence supporting its key role in the disease, including familial forms of PD resulting from mutations in the SNCA gene. Elevated monomeric α-syn levels are also known to cause apoptosis-inducing aggregation in neurons. Additionally, oligomeric forms of α-syn and other IDPs have recently been shown to directly inhibit the proteasome, further disrupting its ability to regulate IDPs concentrations. These data collectively suggest that the accumulation of α-syn and formation of oligomeric species of the IDP play a critical role in the progression of PD. Due to a lack of defined binding pockets, IDPs such as α-syn, and their aggregation are difficult to target through traditional small molecule drug design. There are currently no effective treatments to hinder the progression of neurodegenerative diseases that are associated with IDP accumulation.

### BRIEF DESCRIPTION OF THE FIGURES

The drawings illustrate generally, by way of example, but not by way of limitation, various embodiments discussed herein.

FIG. 1 is concentration response plots (0-40 μM) form fluorogenic peptide digestions with compounds 2, 10, 17, and 18. Error bars denote standard deviation. These data were collected in triplicate.

FIG. 2 shows in invitro digestion of purified alpha-synuclein with human 20S proteasome and enhancement by compound 18. Representative silver stain of these alpha-synuclein digestions and quantification of three trials. 20S proteasome subunits were used as a loading control in the quantification (right). These data were collected in triplicate. Error bars denote standard deviation. Ordinary one-way ANOVA statistical analysis was used to determine statistical significance (ns=not significant, *p<0.05, **p<0.01, **p<0.001, and ****p<0.00001).

### SUMMARY

The disclosure relates to small molecules that enhance proteasome function and restore the activity of impaired proteasomes. Small molecule proteasome enhancers prevent the toxic accumulation of aggregation-prone proteins and prevent neuronal cell death caused by aggregation-prone proteins. The disclosure therefore relates to the use of small molecules as therapeutic agents to treat neurodegenerative diseases. Neurodegenerative diseases include, but are not limited to, Alzheimer's disease (AD) and other dementias, Parkinson's disease (PD) and PD-related disorders, Prion disease, Motor neuron diseases (MND), Huntington's disease (HD), Spinocerebellar ataxia (SCA), Spinal muscular atrophy (SMA).

### DESCRIPTION

Currently, there are no available therapeutics to prevent or slow down the progression of neurodegenerative diseases, such as Alzheimer's and Parkinson's disease.

The disclosure relates to, among other things, compounds that prevent or slow down the progression of neurodegenerative diseases. The disclosure therefore relates to compounds of the formula (I):

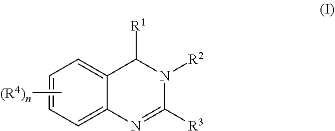

or a pharmaceutically acceptable salt, polymorph, prodrug, solvate or clathrate thereof, wherein:
R¹ is aryl or heteroaryl;
R² is alkyl substituted with cyano; alkoxy; alkoxyalkyl; aryl; (C₄-C₁₀)alkenyl; cycloalkylalkyl; alkynyl; arylalkyl; substituted heteroaryl; (C₂-C₄)heteroaryl or heterocyclylalkyl;
R³ is aryl, cycloalkyl, (C₅-C₁₀)alkyl;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein); and
n is an integer from 1 to 3. For example, each R⁴ is, independently, H, alkyl or alkoxy.

An example of a compound of formula (I) is a compound of the formula (Ia):

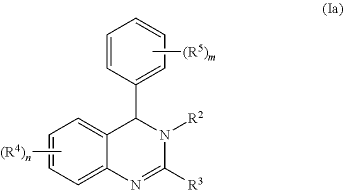

or a pharmaceutically acceptable salt, polymorph, prodrug, solvate or clathrate thereof, wherein:
R² is alkyl substituted with cyano; alkoxy; alkoxyalkyl; aryl; (C₄-C₁₀)alkenyl; cycloalkylalkyl; alkynyl; arylalkyl; substituted heteroaryl; (C₂-C₄)heteroaryl or heterocyclylalkyl;
R³ is aryl, cycloalkyl, (C₅-C₁₀)alkyl;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
m is an integer from 1 to 3; and
n is an integer from 1 to 3. For example, each R⁴ is, independently, H, alkyl or alkoxy.

Another example of a compound of formula (I) is a compound of the formula (Ib):

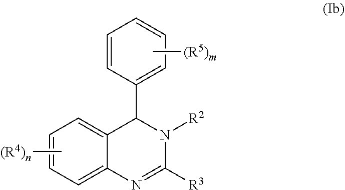

or a pharmaceutically acceptable salt, polymorph, prodrug, solvate or clathrate thereof, wherein:
R² is alkyl substituted with cyano; alkoxy; alkoxyalkyl; aryl; (C₄-C₁₀)alkenyl; cycloalkylalkyl; alkynyl; arylalkyl; substituted heteroaryl; (C₂-C₄)heteroaryl or heterocyclylalkyl;
R³ is aryl, cycloalkyl, (C₅-C₁₀)alkyl;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
m is an integer from 1 to 3; and
n is an integer from 1 to 3. For example, each R⁴ is, independently, H, alkyl or alkoxy.

Another example of a compound of the formula (I) is a compound of the formula (Ic):

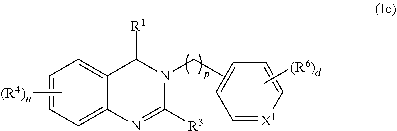

or a pharmaceutically acceptable salt, polymorph, prodrug, solvate or clathrate thereof, wherein:
X¹ is N or CH;
R¹ is aryl or heteroaryl;
R³ is aryl, cycloalkyl, (C₅-C₁₀)alkyl
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein); and
each R₆ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein) or two R⁶ groups on adjacent carbon atoms, together with the carbon atoms to which they are attached, form an aryl or heterocyclyl group;
n and d are each, independently, an integer from 1 to 3; and
p is an integer from 0 to 10, with the proviso that p is not 0 when X¹ is N and R⁶ is H. For example, each R⁴ is, independently, H, alkyl or alkoxy.

Another example of a compound of the formula (I) is a compound of the formula (Id):

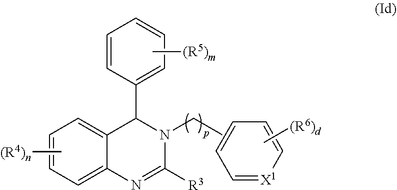

or a pharmaceutically acceptable salt, polymorph, prodrug, solvate or clathrate thereof, wherein:
X¹ is N or CH;
R³ is aryl, cycloalkyl, (C₅-C₁₀)alkyl
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁶ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein) or two R⁶ groups on adjacent carbon atoms, together with the carbon atoms to which they are attached, form an aryl or heterocyclyl group;
m, n, and d are each, independently, an integer from 1 to 3; and
p is an integer from 0 to 10, with the proviso that p is not 0 when X¹ is N and R⁶ is H. For example, each R⁴ is, independently, H, alkyl or alkoxy.

Another example of a compound of the formula (I) is a compound of the formula (Ie):

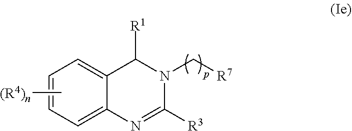

or a pharmaceutically acceptable salt, polymorph, prodrug, solvate or clathrate thereof, wherein:
R¹ is aryl or heteroaryl;
R³ is aryl, cycloalkyl, (C₅-C₁₀)alkyl;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
R⁷ is cycloalkyl or heterocyclylalkyl; n is an integer from 1 to 3; and
p is an integer from 0 to 10. For example, each R⁴ is, independently, H, alkyl or alkoxy.

Another example of a compound of the formula (I) is a compound of the formula (If):

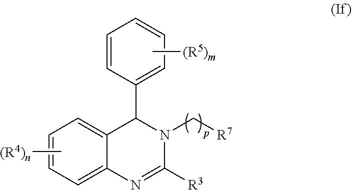

or a pharmaceutically acceptable salt, polymorph, prodrug, solvate or clathrate thereof, wherein:
R³ is aryl, cycloalkyl, (C₅-C₁₀)alkyl;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
R⁷ is cycloalkyl or heterocyclylalkyl; m and n are each, independently, an integer from 1 to 3; and
p is an integer from 0 to 10, with the proviso that R⁷ is not:

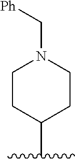 [SMILES: *C1CCN(Cc2ccccc2)CC1]

when p is 0. For example, each R⁴ is, independently, H, alkyl or alkoxy.

Another example of a compound of the formula (I) is a compound of the formula (Ig):

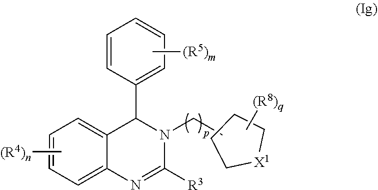

or a pharmaceutically acceptable salt, polymorph, prodrug, solvate or clathrate thereof, wherein:
X² is absent, CH₂, CH₂CH₂, O, OCH₂, NR⁹, wherein R⁹ is H or alkyl, or CH₂NR⁹;
R³ is aryl, cycloalkyl, (C₅-C₁₀)alkyl;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁸ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
m and n are each, independently, an integer from 1 to 3;
q is an integer from 1 to 2; and
p is an integer from 0 to 10, with the proviso that R⁷ is not:

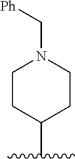 [SMILES: *C1CCN(Cc2ccccc2)CC1]

when p is 0. For example, each R⁴ is, independently, H, alkyl or alkoxy.

In the compounds of the formulae (I) and (Ia)-(Ig), R¹ can be unsubstituted aryl (such as phenyl) or an aryl group substituted with any suitable substituent. Alternatively, or in addition, R², in the compounds of the formulae (I) and (Ia)-(Ig), can be unsubstituted arylalkyl (such as benzyl) or an arylalkyl group substituted with any suitable substituent, whether on the “aryl” portion, the “alkyl” portion or both; or R², in the compounds of the formulae (I) and (Ia)-(Ig), can be unsubstituted heteroaryl (such as quinolinyl or other than 3-pyridinyl) or (C₂-C₄)heteroaryl (such as thiophenyl, pyrrolyl, imidazolyl, triazolyl, and the like) or a heteroaryl group substituted with any suitable substituent; R², in the compounds of the formulae (I) and (Ia)-(Ig), can be an unsubstituted heterocyclylalkyl (such as pyridinylalkyl, piperidinylalkyl or pyranylalkyl) or a heterocyclylalkyl group substituted with any suitable substituent, whether on the “heterocyclyl” portion, the “alkyl” portion or both; R², in the compounds of the formulae (I) and (Ia)-(Ig), can be an unsubstituted cycloalkyl (such as cyclopentyl or cyclohexyl) or can be cycloalkyl substituted with any suitable substituent; or R², in the compounds of the formulae (I) and (Ia)-(Ig), can be an unsubstituted cycloalkylalkyl or cycloalkylalkyl substituted with any suitable substituent, whether on the “cycloalkyl” portion, the “alkyl” portion or both. Alternatively, or in addition, in the compounds of the formulae (I) and (Ia)-(Ig), can be unsubstituted aryl (such as phenyl) or an aryl group substituted with any suitable substituent; R³ in the compounds of the formulae (I) and (Ia)-(Ig), can be unsubstituted (C₅-C₁₀)alkyl or (C₅-C₁₀)alkyl substituted with any suitable substituent; or R³, in the compounds of the formulae (I) and (Ia)-(Ig), can be an unsubstituted cycloalkyl (such as R³ is (C₃-C₆)cycloalkyl, including cyclopentyl and cyclohexyl) or can be cycloalkyl substituted with any suitable substituent. Alternatively, or in addition, R⁴, R⁵, R⁶, and R⁸, in the compounds of the formulae (I) and (Ia)-(Ig), can be any suitable substituent, such as alkyl, alkoxy, and cyano or, in the case of R⁶, two R⁶ groups on adjacent carbon atoms, together with the carbon atoms to which they are attached, form an aryl or heterocyclyl group, thereby forming groups such as naphthyl (when X¹ is CH) and quinolinyl groups (when X¹ is N). Alternatively, or in addition, R⁷ in the compounds of the formulae (I) and (Ia)-(Ig), can be an unsubstituted cycloalkyl (such as cyclopentyl or cyclohexyl) or can be cycloalkyl substituted with any suitable substituent; or R⁷, in the compounds of the formulae (I) and (Ia)-(Ig), can be heterocyclylalkyl (such as pyridinylalkyl, piperidinylalkyl or pyranylalkyl) or a heterocyclylalkyl group substituted with any suitable substituent, whether on the “heterocyclyl” portion, the “alkyl” portion or both. Alternatively, or in addition, X¹ and X² in the compounds of the formulae (I) and (Ia)-(Ig), can independently be N; or in the compounds of the formulae (I) and (Ia)-(Ig), can independently be O.

Examples of compounds of the formulae (I) and (Ia)-(Ig) include, but are not limited to, compounds of the formulae:

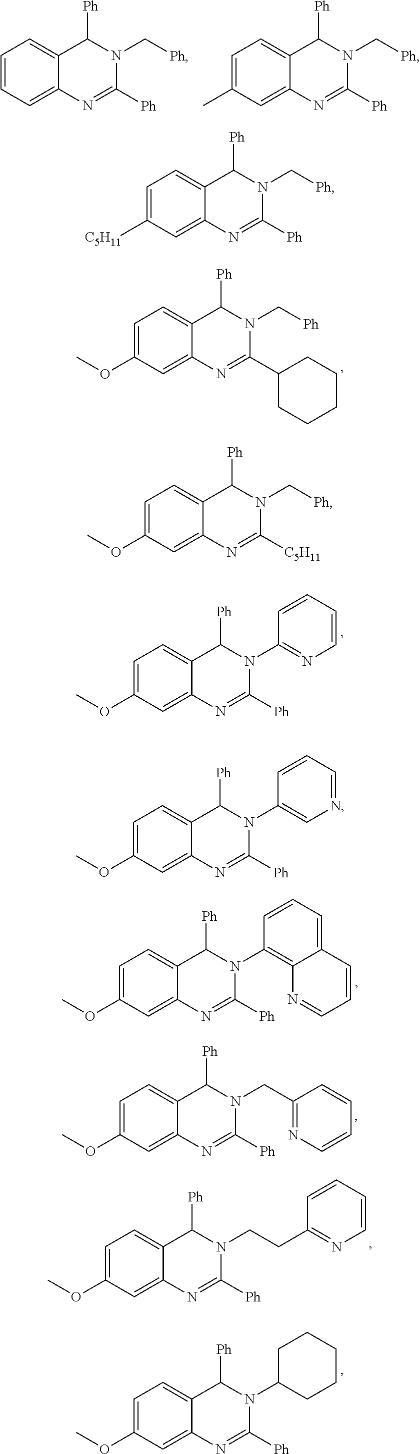

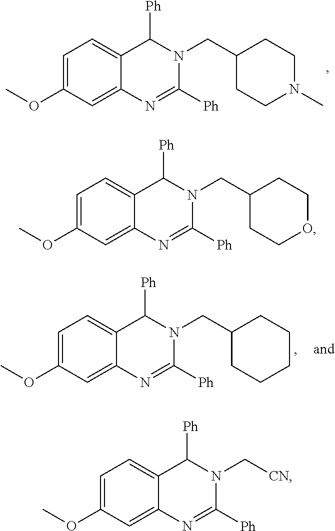

or
pharmaceutically acceptable salts, polymorphs, prodrugs, solvates or clathrates thereof.

This disclosure also relates to a compound of the formula (Ih):

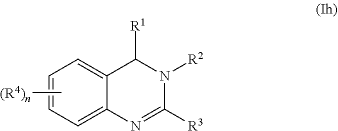

or a pharmaceutically acceptable salt, polymorph, prodrug, solvate or clathrate thereof, wherein:
R¹ is aryl or heteroaryl;
R² and R³, together with the atoms to which they are attached, form a heterocyclic ring;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein); and
n is an integer from 1 to 3.

The heterocyclic ring that can be formed by R² and R³ can be a C₄-C₁₀-heterocyclic ring, such as a saturated C₄-C₁₀-heterocyclic ring.

Examples of compounds of the formula (Ih) include, but are not limited to,

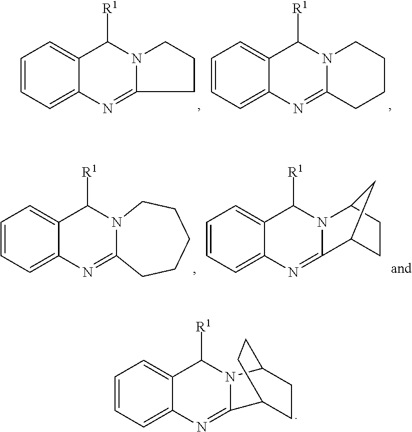

This disclosure also contemplates pharmaceutical compositions comprising one or more compounds and one or more pharmaceutically acceptable excipients. A “pharmaceutical composition” refers to a chemical or biological composition suitable for administration to a subject (e.g., mammal). Such compositions can be specifically formulated for administration via one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” is a carrier, sometimes a liquid, in which an active therapeutic agent is formulated. The excipient generally does not provide any pharmacological activity to the formulation, though it can provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes, but is not limited to, any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, the compounds described herein can be formulated in a time release formulation, for example in a composition that includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical compositions of the present invention can be orally administered as a capsule (hard or soft), tablet (film coated, enteric coated or uncoated), powder or granules (coated or uncoated) or liquid (solution or suspension). The formulations can be conveniently prepared by any of the methods well-known in the art. The pharmaceutical compositions of the present invention can include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, mufti-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestbles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinypyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition according to various embodiments of the present invention can vary according to factors such as the disease state, age, gender, weight, patient history, risk factors, predisposition to disease, administration route, pre-existing treatment regime (e.g., possible interactions with other medications), and weight of the subject. Dosage regimens can be adjusted to provide the optimum therapeutic response. For example, a single bolus can be administered, several divided doses can be administered over time, or the dose can be proportionally reduced or increased as indicated by the exigencies of therapeutic situation.

A “dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in subjects. In therapeutic use for treatment of conditions in mammals (e.g., humans) for which the compounds of the present invention or an appropriate pharmaceutical composition thereof are effective, the compounds of the present invention can be administered in an effective amount. The dosages as suitable for this invention can be a composition, a pharmaceutical composition or any other compositions described herein.

For each of the recited embodiments, the dosage is typically administered once, twice, or thrice a day, although more frequent dosing intervals are possible. The dosage can be administered every day, every 2 days, every 3 days, every 4 days, every 5 days, every 6 days, and/or every 7 days (once a week). In one embodiment, the dosage can be administered daily for up to and including 30 days, preferably between 7-10 days. In another embodiment, the dosage can be administered twice a day for 10 days. If the patient requires treatment for a chronic disease or condition, the dosage can be administered for as long as signs and/or symptoms persist. The patient can require “maintenance treatment” where the patient is receiving dosages every day for months, years, or the remainder of their lives. In addition, the composition of this invention can be to effect prophylaxis of recurring symptoms. For example, the dosage can be administered once or twice a day to prevent the onset of symptoms in patients at risk, especially for asymptomatic patients.

The absolute weight of a given compound included in a unit dose for administration to a subject can vary widely. For example, about 0.0001 to about 1 g, or about 0.001 to about 0.5 g, of at least one compound of this disclosure, or a plurality of compounds can be administered. Alternatively, the unit dosage can vary from about 0.001 g to about 2 g, from about 0.005 g to about 0.5 g, from about 0.01 g to about 0.25 g, from about 0.02 g to about 0.2 g, from about 0.03 g to about 0.15 g, from about 0.04 g to about 0.12 g, or from about 0.05 g to about 0.1 g.

Daily doses of the compounds can vary as well. Such daily doses can range, for example, from about 0.01 g/day to about 10 g/day, from about 0.02 g/day to about 5 g/day, from about 0.03 g/day to about 4 g/day, from about 0.04 g/day to about 3 g/day, from about 0.05 g/day to about 2 g/day, and from about 0.05 g/day to about 1 g/day.

It will be appreciated that the amount of compound(s) for use in treatment will vary not only with the particular carrier selected but also with the route of administration, the nature of the condition being treated, and the age and condition of the patient. Ultimately the attendant health care provider may determine proper dosage.

The compositions described herein can be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to, for example, tissue, organ, and/or organ system, which encompasses and/or is affected by the disease, and/or where the disease signs and/or symptoms are active or are likely to occur. Administration can be topical with a local effect, composition is applied directly where its action is desired. Administration can be enteral wherein the desired effect is systemic (non-local), composition is given via the digestive tract. Administration can be parenteral, where the desired effect is systemic, composition is given by other routes than the digestive tract.

The compositions can include the compounds described herein in a “therapeutically effective amount.” Such a therapeutically effective amount is an amount sufficient to obtain the desired physiological effect, such as a reduction of at least one symptom of cancer or an inflammatory disease or condition.

The compositions contemplated herein can contain other ingredients such as chemotherapeutic agents, anti-inflammatory agents, anti-viral agents, antibacterial agents, antimicrobial agents, immunomodulatory drugs, such as lenalidomide, pomalidomide or thalidomide, histone deacetylase inhibitors, such as panobinostat, preservatives or combinations thereof.

This disclosure also includes methods for treating neurodegenerative diseases, such as Parkinson's disease, Alzheimer's disease, Huntington's disease, and ALS, comprising administering a therapeutically effective amount of at least one of the compounds described herein (e.g., compounds of the formulae (I) and (Ia)-(Ih)) to a subject in need thereof. This disclosure also includes methods for reducing, substantially eliminating or eliminating dysregulation of proteostasis comprising administering a therapeutically effective amount of at least one of the compounds described herein (e.g., compounds of the formulae (I), (Ia)-(Ih)) to a subject in need thereof. This disclosure also includes methods for reducing, substantially eliminating or eliminating the accumulation of intrinsically disordered proteins (e.g., α-syn) comprising administering a therapeutically effective amount of at least one of the compounds described herein (e.g., compounds of the formulae (I), (Ia)-(Ig)) to a subject in need thereof.

As used herein, the terms “treat” and “treating” are not limited to the case where the subject (e.g. patient) is cured and the disease is eradicated. Rather, treatment that merely reduces symptoms, and/or delays disease progression is also contemplated.

The pharmaceutical compositions disclosed herein can have the ability to effectively treat new patient segments where proteasome inhibition and reduced toxicity is desired or warranted.

The compounds and methods described herein can be used prophylactically or therapeutically. The term “prophylactic” or “therapeutic” treatment refers to administration of a drug to a host before or after onset of a disease or condition. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom). Administering the compounds described herein (including enantiomers and salts thereof) is contemplated in both a prophylactic treatment (e.g. to patients at risk for disease, such as elderly patients who, because of their advancing age, are at risk for arthritis, cancer, and the like) and therapeutic treatment (e.g. to patients with symptoms of disease or to patients diagnosed with disease).

The term “therapeutically effective amount” as used herein, refers to that amount of one or more compounds of the various examples of the present invention that elicits a biological or medicinal response in a tissue system, animal or human, that is being sought by a researcher, veterinarian, medical doctor or other clinician, which includes alleviation of the symptoms of the disease or disorder being treated. In some examples, the therapeutically effective amount is that which can treat or alleviate the disease or symptoms of the disease at a reasonable benefit/risk ratio applicable to any medical treatment. However, it is to be understood that the total daily usage of the compounds and compositions described herein can be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds described herein.

The term “alkyl” as used herein refers to substituted or unsubstituted straight chain, branched and cyclic, saturated mono- or bi-valent groups having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. Examples of straight chain bi-valent (C₁-C₂₀)alkyl groups include those with from 1 to 6 carbon atoms such as —CH₂—, —CH₂CH₂—, —CH₂CH₂CH₂—, —CH₂CH₂CH₂CH₂—, and —CH₂CH₂CH₂CH₂CH₂—. Examples of branched bi-valent alkyl groups include —CH(CH₃)CH₂— and —CH₂CH(CH₃)CH₂—. Examples of cyclic alkyl groups include cyclopropyl, cyclobutyl, cyclopently, cyclohexyl, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. In some embodiments, alkyl includes a combination of substituted and unsubstituted alkyl. As an example, alkyl, and also (C₁)alkyl, includes methyl and substituted methyl. As a particular example, (C₁)alkyl includes benzyl. As a further example, alkyl can include methyl and substituted (C₂-C₈)alkyl. Alkyl can also include substituted methyl and unsubstituted (C₂-C₈)alkyl. In some embodiments, alkyl can be methyl and C₂-C₈ linear alkyl. In some embodiments, alkyl can be methyl and C₂-C₈ branched alkyl. The term methyl is understood to be —CH₃, which is not substituted. The term methylene is understood to be —CH₂—, which is not substituted. For comparison, the term (C₁)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further example, representative substituted alkyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkyl groups can be substituted from a set of groups including amino, hydroxy, cyano, carboxy, nitro, thio and alkoxy, but not including halogen groups. Thus, in some embodiments alkyl can be substituted with a non-halogen group. For example, representative substituted alkyl groups can be substituted with a fluoro group, substituted with a bromo group, substituted with a halogen other than bromo, or substituted with a halogen other than fluoro. In some embodiments, representative substituted alkyl groups can be substituted with one, two, three or more fluoro groups or they can be substituted with one, two, three or more non-fluoro groups. For example, alkyl can be trifluoromethyl, difluoromethyl, or fluoromethyl, or alkyl can be substituted alkyl other than trifluoromethyl, difluoromethyl or fluoromethyl. Alkyl can be haloalkyl or alkyl can be substituted alkyl other than haloalkyl. The term “alkyl” also generally refers to alkyl groups that can comprise one or more heteroatoms in the carbon chain. Thus, for example, “alkyl” also encompasses groups such as —[(CH₂)ₚO]_{q}H and the like.

The term “alkenyl” as used herein refers to substituted or unsubstituted straight chain, branched and cyclic, saturated mono- or bi-valent groups having at least one carbon-carbon double bond and from 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. The double bonds can be trans or cis orientation. The double bonds can be terminal or internal. The alkenyl group can be attached via the portion of the alkenyl group containing the double bond, e.g., vinyl, propen-1-yl and buten-1-yl, or the alkenyl group can be attached via a portion of the alkenyl group that does not contain the double bond, e.g., penten-4-yl. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl. Examples of straight chain bi-valent (C₂-C₂₀)alkenyl groups include those with from 2 to 6 carbon atoms such as —CHCH—, —CHCHCH₂—, —CHCHCH₂CH₂—, and —CHCHCH₂CH₂CH₂—. Examples of branched bi-valent alkyl groups include —C(CH₃)CH— and —CHC(CH₃)CH₂—. Examples of cyclic alkenyl groups include cyclopentenyl, cyclohexenyl and cyclooctenyl. It is envisaged that alkenyl can also include masked alkenyl groups, precursors of alkenyl groups or other related groups. As such, where alkenyl groups are described it, compounds are also envisaged where a carbon-carbon double bond of an alkenyl is replaced by an epoxide or aziridine ring. Substituted alkenyl also includes alkenyl groups which are substantially tautomeric with a non-alkenyl group. For example, substituted alkenyl can be 2-aminoalkenyl, 2-alkylaminoalkenyl, 2-hydroxyalkenyl, 2-hydroxyvinyl, 2-hydroxypropenyl, but substituted alkenyl is also understood to include the group of substituted alkenyl groups other than alkenyl which are tautomeric with non-alkenyl containing groups. In some embodiments, alkenyl can be understood to include a combination of substituted and unsubstituted alkenyl. For example, alkenyl can be vinyl and substituted vinyl. For example, alkenyl can be vinyl and substituted (C₃-C₈)alkenyl. Alkenyl can also include substituted vinyl and unsubstituted (C₃-C₈)alkenyl. Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further example, representative substituted alkenyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkenyl groups can be substituted from a set of groups including monoalkylamino, dialkylamino, cyano, acetyl, amido, carboxy, nitro, alkylthio and alkoxy, but not including halogen groups. Thus, in some embodiments alkenyl can be substituted with a non-halogen group. In some embodiments, representative substituted alkenyl groups can be substituted with a fluoro group, substituted with a bromo group, substituted with a halogen other than bromo, or substituted with a halogen other than fluoro. For example, alkenyl can be 1-fluorovinyl, 2-fluorovinyl, 1,2-difluorovinyl, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. In some embodiments, representative substituted alkenyl groups can be substituted with one, two, three or more fluoro groups or they can be substituted with one, two, three or more non-fluoro groups.

The term “alkynyl” as used herein, refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. Examples include, but are not limited to ethynyl, propynyl, propyn-1-yl, propyn-2-yl, butynyl, butyn-1-yl, butyn-2-yl, butyn-3-yl, butyn-4-yl, pentynyl, pentyn-1-yl, hexynyl, Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀)aryl encompasses mono- and polycyclic (C₆-C₂₀)aryl groups, including fused and non-fused polycyclic (C₆-C₂₀)aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₈) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

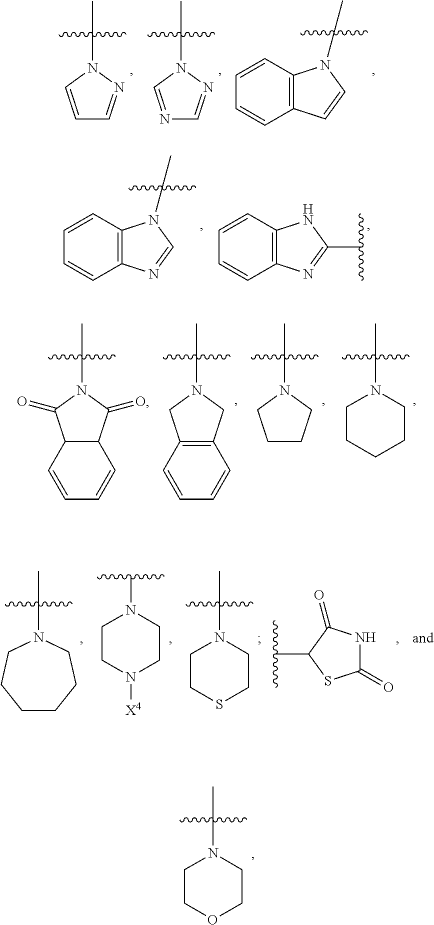

wherein X₄ represents H, (C₁-C₂₀)alkyl, (C₆-C₂₀)aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. Thus, alkyoxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryloxy” as used herein refers to an oxygen atom connected to an aryl group as are defined herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The terms “halo,” “halogen,” or “halide” group, as used herein, by themselves or as part of another substituent, mean, unless otherwise stated, a fluorine, chlorine, bromine, or iodine atom.

The term “amine” and “amino” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and triakylamino group.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, aryl, cycloalkyl, heterocyclyl, group or the like.

The term “formyl” as used herein refers to a group containing a carbonyl moiety wherein the group is bonded via the carbonyl carbon atom. The carbonyl carbon atom is also bonded to a hydrogen atom.

The term “alkoxycarbonyl” as used herein refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom is also bonded to an oxygen atom which is further bonded to an alkyenyl group. Alkoxycarbonyl also includes the group where a carbonyl carbon atom is also bonded to an oxygen atom which is further bonded to an alkynyl group. In a further case, which is included in the definition of alkoxycarbonyl as the term is defined herein, and is also included in the term “aryloxycarbonyl,” the carbonyl carbon atom is bonded to an oxygen atom which is bonded to an aryl group instead of an alkyl group.

The term “arylcarbonyl” as used herein refers to a group containing a carbonyl moiety wherein the group is bonded via the carbonyl carbon atom. The carbonyl carbon atom is also bonded to an aryl group.

The term “alkylamido” as used herein refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylamido as the term is defined herein, the carbonyl carbon atom is bonded to a nitrogen atom which is bonded to one or more aryl group instead of, or in addition to, the one or more alkyl group. In a further case, which is also an alkylamido as the term is defined herein, the carbonyl carbon atom is bonded to an nitrogen atom which is bonded to one or more alkenyl group instead of, or in addition to, the one or more alkyl and or/aryl group. In a further case, which is also an alkylamido as the term is defined herein, the carbonyl carbon atom is bonded to a nitrogen atom which is bonded to one or more alkynyl group instead of, or in addition to, the one or more alkyl, alkenyl and/or aryl group.

The term “carboxy” as used herein refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “amido” or “amide” as used herein refers to a group having the formula C(O)NRR, wherein R is defined herein and can each independently be, e.g., hydrogen, alkyl, aryl or each R, together with the nitrogen atom to which they are attached, form a heterocyclyl group.

The term “alkylthio” as used herein refers to a sulfur atom connected to an alkyl, alkenyl, or alkynyl group as defined herein.

The term “arylthio” as used herein refers to a sulfur atom connected to an aryl group as defined herein.

The term “alkylsulfonyl” as used herein refers to a sulfonyl group connected to an alkyl, alkenyl, or alkynyl group as defined herein.

The term “alkylsulfinyl” as used herein refers to a sulfinyl group connected to an alkyl, alkenyl, or alkynyl group as defined herein.

The term “dialkylaminosulfonyl” as used herein refers to a sulfonyl group connected to a nitrogen further connected to two alkyl groups, as defined herein, and which can optionally be linked together to form a ring with the nitrogen. This term also includes the group where the nitrogen is further connected to one or two alkenyl groups in place of the alkyl groups.

The term “dialkylamino” as used herein refers to an amino group connected to two alkyl groups, as defined herein, and which can optionally be linked together to form a ring with the nitrogen. This term also includes the group where the nitrogen is further connected to one or two alkenyl groups in place of the alkyl groups.

The term “dialkylamido” as used herein refers to an amido group connected to two alkyl groups, as defined herein, and which can optionally be linked together to form a ring with the nitrogen. This term also includes the group where the nitrogen is further connected to one or two alkenyl groups in place of the alkyl groups.

The term “substituted” as used herein refers to a group that is substituted with one or more groups including, but not limited to, the following groups: halogen (e.g., F, Cl, Br, and I), R, OR, ROH (e.g., CH₂OH), OC(O)N(R)₂ (also known as carbamate), CN, NO, NO₂, ONO₂, azido, CF₃, OCF₃, methylenedioxy, ethylenedioxy, (C₃-C₂₀)heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(—NH)N(R)₂, C(O)N(OR)R, or C(—NOR)R wherein R can be hydrogen, (C₁-C₂₀)alkyl, (C₆-C₂₀)aryl, heterocyclyl or polyalkylene oxide groups, such as polyalkylene oxide groups of the formula —(CH₂CH₂O)ₜ—R—OR, —(CH₂CH₂CH₂O)_{g}—R—OR, —(CH₂CH₂O)_{f}(CH₂CH₂CH₂O)_{g}—R—OR each of which can, in turn, be substituted or unsubstituted and wherein f and g are each independently an integer from 1 to 50 (e.g., 1 to 10, 1 to 5, 1 to 3 or 2 to 5). Substituted also includes a group that is substituted with one or more groups including, but not limited to, the following groups: fluoro, chloro, bromo, iodo, amino, amido, alkyl, hydroxy, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

In some instances, the compounds described herein (e.g., compounds of the formulae (I) and (Ia)-(Ig)) can contain chiral centers. All diastereomers of the compounds described herein are contemplated herein, as well as racemates.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

As used herein, the term “subject” or “patient” refers to any organism to which a composition described herein can be administered, e.g., for experimental, diagnostic, prophylactic and/or therapeutic purposes. Subject refers to a mammal receiving the compositions disclosed herein or subject to disclosed methods. It is understood and herein contemplated that “mammal” includes but is not limited to humans, non-human primates, cows, horses, dogs, cats, mice, rats, rabbits, and guinea pigs.

Each embodiment described above is envisaged to be applicable in each combination with other embodiments described herein. For example, embodiments corresponding to formula (I) are equally envisaged as being applicable to formulae (Ia)-(Ig).

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed can be resorted to by those of ordinary skill in the art, and that such modifications and variations are considered to be within the scope of embodiments of the present disclosure

The invention is now described with reference to the following Examples. The following working examples therefore, are provided for the purpose of illustration only and specifically point out certain embodiments of the present invention, and are not to be construed as limiting in any way the remainder of the disclosure. Therefore, the examples should be construed to encompass any and all variations which become evident as a result of the teaching provided herein.

### EXAMPLES

The present disclosure can be better understood by reference to the following examples which are offered by way of illustration. The disclosure is not limited to the examples given herein.

Generally speaking, the synthesis of dihydroquinazolines such as the compounds of the formulae (I) and (Ia)-(Ig), is accomplished via a one-pot multicomponent reaction of amides amines and aldehydes (Scheme 1) This work is described in Bioorg. Med. Chem. Lett. 36: 127821 (2021) and Org. Biomol. Chem 17: 7995 (2019), both of which are incorporated by reference as if fully set forth herein.

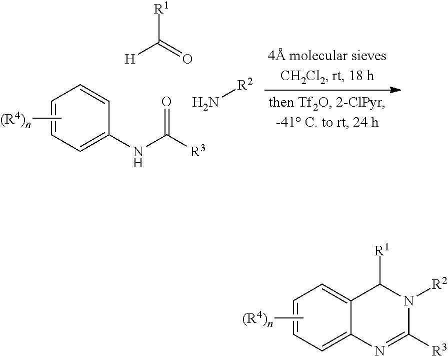

This method involves in situ imine formation from an amine and an aldehyde in the presence of molecular sieves, followed by tandem assembly of the heterocyclic ring through successive Tf₂O-mediated amide dehydration, imine insertion, and Pictet-Spengler-like cyclization. The multicomponent nature of the method permits the construction of highly diverse dihydroquinazolines due to the compatibility of a wide range of simple starting materials. A library of dihydroquinazolines was generated using the multicomponent method to probe the ability of members of this class of compounds to activate the 20S proteasome. Compounds chosen to populate the library differed in the ˜ structural features at the 7-, 0.2-, and 3-positions (R⁴, R³ and R², respectively) of the heterocyclic scaffold. Variation at the 7- and 2-positions was accomplished using select amides, while the substituents at the 3-position were introduced using chosen amines. R³ and R⁴ groups introduced from the starting amides provided preliminary structure activity relationship information which was utilized for the construction of the remaining members of the compound library in which the R² group was varied. Simple alkyl and alkoxy substituents were explored at R⁴, along with the absence of any additional group at this location, and the investigated R³ substituents included alkyl and cycloalkyl groups to compare them to the aromatic counterpart. A range of R² substituents were installed to include aryl and heteroaryl groups, tethered heteroaryl groups, and alkyl groups with varying ring and heteroatom placement.

### Experimental Procedures

Materials and Reagents. Human 20S proteasome and fluorogenic substrates N-succinyl-Leu-Leu-Val-Tyr-7-amido-4-methylcoumarin (Suc-LLVY-AMC), carboxyl benzyl-Leu-Leu-Glu-7-amido-4-methylcoumarin (Z-LLE-AMC), tert-butyloxycarbonyl-Leu-Arg-Arg-7-amido-4-methylcoumarin (Boc-LRR-AMC), and bortezomib were obtained from Boston Biochem, Inc. (Cambridge, MA). The PVDF membrane, Clarity western ECL reagent, blocking grade milk, and precast sodium dodecyl sulfate (SDS) gels were from Bio-Rad (Hercules, CA). The recombinant wild type α-synuclein was obtained from Abcam (Cambridge, MA). Mouse monoclonal anti-α-synuclein and anti-mouse HRP were purchased from Santa Cruz Biotechnologies (Dallas, TX).

Unless otherwise noted, chemicals were purchased from commercial suppliers and used without further purification. CH₂Cl₂ was distilled under N₂ from CaH₂, and 2-chloropyridine was dried over 4 Å molecular sieves. Amide N-(3-methoxyphenyl)benzamide and dihydroquinazolines 2, 6, 7, 9, 10, 12, 18, 23, and 24 were prepared using known methods known in the art.

### Fluorogenic Peptide Degradation 20S Proteasome Activity Assay.

Activity assays were carried out in a 100 μL reaction volume. Different concentrations (40-1.25 μM) of test compounds (1 μL) were added to a black flat/clear bottom 96-well plate containing 2 nM of human constitutive 20S proteasome (89 μL), in 50 mM Tris-HCl at pH 7.8, 100 mM NaCl and allowed to incubate for 15 min at 37° C. Fluorogenic substrates (5 μL) were then added and the enzymatic activity measured at 37° C. on a SpectraMax M5e spectrometer by measuring the change in fluorescence unit per minute for 1 hour at 380-460 nm. The fluorescence units for the vehicle control were set at a 100%, and the ratio of drug-treated sample set to that of vehicle control was used to calculate the fold change in enzymatic activity. The fluorogenic substrates used were one of the following: Suc-LLVY-AMC (CT-L activity, 20 μM), Z-LLE-AMC (Casp-L activity, 20 μM), Boc-LRR-AMC (Trp-L activity, 40 μM) or a combination of the three substrates each at 6.67 μM. Results were analysed and plotted using GraphPad Prism 7.

In vitro purified α-synuclein degradation assay. Digestion of α-synuclein was carried out in a 50 μL reaction volume made of 50 mM Tris and 100 mM NaCl at pH 7.8; 0.5 μM purified α-synuclein and 6.7 nM purified human 20S proteasome. Briefly, 20S proteasome was diluted to 7.58 nM in the reaction buffer. Test compounds or vehicle (1 μL of 50× stock or DMSO) were added to 44 μL of 7.58 nM 20S and incubated at 37° C. for 20 min. 5 μL of 5 μM α-synuclein substrate was then added to the reaction mixture and incubated at 37° C. for 4 hours. The reactions were quenched with concentrated sodium dodecyl sulfate (SDS) loading buffer. After boiling for 10 min, samples were resolved on a 4-20% Tris-glycine SDS-PAGE gel. The gels were then stained using a Pierce Silver Stain Kit (Thermo Scientific, Rockford IL) and the provided procedure. Silver stained gels were then quantified using ImageJ.

General Experimental Information. Reactions were carried out in flame-dried glassware under nitrogen atmosphere. All reactions were magnetically stirred and monitored by TLC on EMD Millipore silica gel 60F₂₅₄ pre-coated glass plates using UV light (254 nm) to visualize the compounds. Column chromatography was carried out on SiliaFlash P60 (230-400 mesh) silica gel supplied by SiliCycle or on a Yamazen AKROS MPLC system using silica gel columns supplied by Yamazen Corporation. Infrared spectra were recorded on an Agilent Technologies Cary 630 FT-IR spectrometer. Proton (¹H NMR) and carbon (¹³C NMR) nuclear magnetic resonance spectra were recorded on a Bruker Avance Ill 400 MHz spectrometer. The chemical shifts are given in parts per million (ppm) on the delta (67) scale. Tetramethylsilane (TMS) or the residual solvent peak was used as a reference value. High resolution mass spectra were recorded at the LSSU Cannabis Center of Excellence on an Agilent 1290 Ultra-High Pressure Liquid Chromatograph with a Time of Flight Mass Spectrometer (UHPLC-TOF). Melting points were obtained using a Mel-Temp capillary melting point apparatus and are uncorrected.

General procedure for amide synthesis: To a mixture of an amine and triethylamine in anhydrous CH₂Cl₂, cooled to 0° C. in an ice bath, was added dropwise an appropriate acid chloride followed by 4-dimethylaminopyridine (4-DMAP). The ice bath was removed, and the reaction stirred at room temperature under N₂ atmosphere until complete, as determined by TLC. The reaction mixture was washed with saturated NaHCO₃ solution (×3) and brine before being dried (Na₂SO₄) and concentrated. The crude product was then purified either by crystallization or by chromatography.

General procedure for dihydroquinazoline synthesis: A mixture of amide, amine, aldehyde, and 4 Å molecular sieves in CH₂Cl₂ was prepared and stirred for 18 h at room temperature under N₂ atmosphere. The reaction mixture was cooled to −41° C. and was treated successively with 2-chloropyridine followed by Tf₂O. The reaction was then allowed to warm to room temperature and was stirred for 24 hours. The molecular sieves were filtered from the reaction, and the filtrate was washed with saturated aqueous NaHCO₃ solution before being dried (Na₂SO₄) and concentrated. The crude mixture was then purified via chromatography.

### Examples 1-21

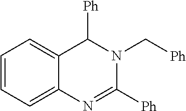 [SMILES: c1ccc(CN2C(c3ccccc3)=Nc3ccccc3C2c2ccccc2)cc1]

3-Benzyl-2,4-diphenyl-3,4-dihydroquinazoline (1). Prepared according to the general dihydroquinazoline protocol with N-phenyl-benzamide (0.396 g, 2.01 mmol), benzylamine (0.24 mL, 2.2 mmol), benzaldehyde (0.22 mL, 2.2 mmol), CH₂Cl₂ (20 mL), 2-chloropyridine (0.225 mL, 2.4 mmol), and Tf₂O (0.37 mL, 2.2 mmol). The reaction proceeded for 48 h at rt after addition of Tf₂O and was worked up as described above. The residue was purified by MPLC (12-50% EtOAc in hexanes as eluent) followed by additional purification via MPLC (20:802 ether:CH₂Cl₂:MeOH as eluent) to afford the desired product (0.098 g, 1% yield) as an oil. ¹H NMR (400 MHz, CDCl₃) 7.55-7.48 (m, 2H), 7.43-7.26 (m, 12H), 7.23-7.16 (m, 3H), 6.94 (td, J=7.4, 1.3 Hz, 1H), 6.73 (dd, J=7.6, 1.3 Hz, 1H), 5.41 (s, 1H), 4.71 (d, J=15.6 Hz, 1H), 4.00 (d, J=15.6 Hz, 1H); ¹³C NMR (101 MHz, CDCl₃) δ 157.9, 143.8, 141.3, 136.61, 136.58, 129.3, 129.1, 128.8, 128.7, 128.3, 128.2, 128.1, 127.8, 127.4, 127.0, 126.4, 125.0, 124.9, 124.8, 60.7, 53.3; IR (neat): 3060, 3029, 1549, 1484, 1454, 1321 cm⁻¹; HRMS (ESI): m/z calcd for C₂₇H₂₃N₂ [M+H], 375.1861; found, 375.1872.

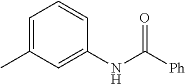 [SMILES: Cc1cccc(NC(=O)c2ccccc2)c1]

N-(3-methylphenyl)benzamide (S1). Prepared according to the general amide protocol using m-toluidine (1.10 mL g, 10.3 mmol), TEA (1.50 mL, 10.8 mmol), benzoyl chloride (1.25 25 mL, 10.8 mmol), 4-DMAP (0.089 g, 0.07 mmol), and EtOAc (50 mL) instead of CH₂Cl₂. The reaction stirred overnight and was worked up as described above with EtOAc used for extractions instead of CH₂Cl₂. The residual solid was purified by crystallization from EtOAc and hexanes to afford the desired product (1.815, 84% yield) as a solid (m.p.=122-124° C.). ¹H NMR (400 MHz, CDCl₃) δ 7.90-7.83 (m, 2H), 7.78 (s, 1H), 7.58-7.45 (m, 4H), 7.41 (dd, J=8.1, 2.1 Hz, 1H), 7.25 (t, J=7.8 Hz, 1H), 6.97 (d, J=7.5 Hz, 1H), 2.37 (s, 3H); ¹³C NMR (101 MHz, CDCl₃) δ 165.6, 139.1, 137.8, 135.1, 131.8, 128.9, 128.8, 127.0, 125.4, 120.8, 117.2, 21.5.

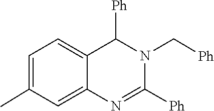 [SMILES: Cc1ccc2c(c1)N=C(c1ccccc1)N(Cc1ccccc1)C2c1ccccc1]

3-Benzyl-7-methyl-2,4-diphenyl-3,4-dihydroquinazoline (3). Prepared according to the general dihydroquinazoline protocol with S1 (0.211 g, 1.00 mmol), benzylamine (0.12 mL, 1.1 mmol), benzaldehyde (0.11 mL, 1.1 mmol), CH₂Cl₂ (10.0 mL), 2-chloropyridine (0.11 mL, 1.2 mmol), and Tf₂O (0.18 mL, 1.1 mmol). The reaction proceeded for 24 h at rt after addition of Tf₂O and was worked up as described above. The residue was passed through a silica plug (90:10:0.5 EtOAc:hexanes:TEA as eluent) and the concentrated filtrate was further purified by flash chromatography (25% EtOAc in hexanes as eluent) to afford the desired product (0.142 g, 37% yield) as a solid (m.p.=175-177° C.). ¹H NMR (400 MHz, CDCl₃) δ 7.53-7.47 (m, 2H), 7.41-7.24 (m, 11H), 7.21-7.13 (m, 3H), 6.76 (dd, J=7.7, 1.1 Hz, 1H), 6.62 (d, J=7.7 Hz, 1H), 5.38 (s, 1H), 4.70 (d, J=15.7 Hz, 1H), 3.99 (d, J=15.7 Hz, 1H), 2.28 (s, 3H); ¹³C NMR (101 MHz, CDCl₃) δ 157.9, 144.0, 141.1, 138.0, 136.67, 136.65, 129.3, 129.0, 128.8, 128.6, 128.1, 128.0, 127.7, 127.3, 126.9, 126.2, 125.8, 125.5, 122.0, 60.6, 53.3, 21.2; IR (neat): 3062, 2920, 1586, 1549, 1493, 1422, 1321 cm⁻¹; HRMS (ESI): m/z calcd for C₂₈H₂₄N₂ [M+H], 389.2018; found, 389.2017.

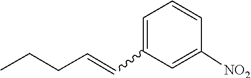

(E2)-1-(m-Nitrophenyl)-1-pentene (S2). A mixture of 3-nitrobenzaldehyde (3.039 g, 20.1 mmol), butyltriphenylphosphonium bromide (9.607 g, 24.0 mmol), and K₂CO₃ (8.25 g, 62.9 mmol) in toluene (110 mL) was heated to reflux overnight. The reaction cooled to room temperature and the reaction was filtered. The filtrate was concentrated under vacuum, and the residue was purified by flash chromatography (5% EtOAc in hexanes as eluent) to afford the desired product (3.436 g, 89% yield) as an oil (˜2:1 mixture of diasteromers). ¹H NMR (400 MHz, CDCl₃) δ 8.09 (t, J=2.0 Hz, 0.3H), 8.06 (t, J=2.1 Hz, 0.7H), 8.01 (ddd, J=8.1, 2.3, 1.1 Hz, 0.7H), 7.95 (ddd, J=8.2, 2.3, 1.1 Hz, 0.3H), 7.61-7.52 (m, 1H), 7.45 (t, J=7.9 Hz, 0.7H), 7.39 (t, J=8.0 Hz, 0.3H), 6.44-6.31 (m, 1.3H), 5.79 (dt, J=11.6, 7.3 Hz, 0.7H), 2.28 (qd, J=7.3, 1.9 Hz, 1.4H), 2.23-2.15 (m, 0.6H), 1.56-1.42 (m, 2H), 0.99-0.89 (m, 3H); ¹³C NMR (101 MHz, CDCl₃) δ 148.7, 148.3, 139.8, 139.4, 135.9, 134.8, 134.4, 131.9, 129.4, 129.1, 128.0, 126.9, 123.3, 121.3, 121.3, 120.4, 35.2, 30.7, 23.1, 22.4, 13.82, 13.80; IR (neat): 3008, 2958, 1525, 1346 cm⁻¹; HRMS (ESI): m/z calcd for C₁₁H₁₄NO₂ [M+H], 192.1025; found, 192.1019.

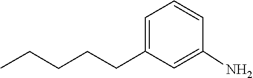

3-Pentylaniline (S3). To a solution of S2 (1.486 g, 7.77 mmol) in EtOH (50 mL) was added 10% Pd/C (0.455 g), and the reaction flask containing the mixture was fitted with a H₂ balloon. The reaction stirred at room temperature for 20 h, after which the reaction mixture was passed through a celite plug with EtOAc. The concentrated was concentrated and subsequently purified via MPLC (7-35% EtOAc in hexanes as eluent) to afford the desired product (1.153 g, 91% yield) as a light orange oil. ¹H NMR (400 MHz, CDCl₃) δ 7.02 (t, J=7.6 Hz, 1H), 6.56 (dt, J=7.4, 1.3 Hz, 1H), 6.49-6.40 (m, 2H), 3.50 (s, 2H), 2.59-2.40 (m, 2H), 1.64-1.50 (m, 2H), 1.39-1.25 (m, 4H), 0.88 (t, J=6.9 Hz, 3H); ¹³C NMR (101 MHz, CDCl₃) δ 146.5, 144.3, 129.2, 118.9, 115.4, 112.6, 36.1, 31.7, 31.2, 22.7, 14.2; IR (neat): 3461, 3364, 3032, 2924, 1618, 1590, 1459, 1288 cm⁻¹; HRMS (ESI): m/z calcd for C₁₁H₁₈N [M+H], 164.1439; found, 164.1437.

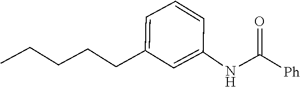 [SMILES: CCCCCc1cccc(NC(=O)c2ccccc2)c1]

N-(3-pentylphenyl)-benzamide (S4). Prepared according to general procedure A for amide synthesis using S3 (0.612 g, 3.17 mmol), TEA (0.52 mL, 3.7 mmol), benzoyl chloride (0.40 mL, 3.4 mmol), 4-DMAP (0.008 g, 0.05 mmol), and CH₂Cl₂ (20 mL). The reaction stirred overnight before being worked up as described. The residue was purified by flash chromatography (10% EtOAc in hexanes as eluent) to afford the desired product (0.759 g, 90% yield) as a solid (m.p.=76-78° C.). ¹H NMR (400 MHz, CDCl₃) δ 8.68 (s, 1H), 7.88 (d, J=7.1 Hz, 2H), 7.62-7.52 (m, 2H), 7.48 (t, J=7.4 Hz, 1H), 7.37 (dd, J=8.4, 7.0 Hz, 2H), 7.23 (t, J=7.8 Hz, 1H), 6.99 (d, J=7.6 Hz, 1H), 2.58 (dd, J=8.8, 6.8 Hz, 2H), 1.63 (p, J=7.4 Hz, 2H), 1.35 (qd, J=6.1, 4.1 Hz, 4H), 0.94 (t, J=6.8 Hz, 3H); ¹³C NMR (101 MHz, CDCl₃) δ 166.2, 143.8, 138.1, 135.0, 131.5, 128.6, 128.4, 127.2, 124.6, 120.6, 118.0, 35.9, 31.5, 31.0, 22.5, 14.0; IR (neat): 3308, 2928, 1649, 1541, 1487, 1316, 1269 cm⁻¹; HRMS (ESI): m/z calcd for C₁₈H₂₂NO [M+H], 268.1701; found, 268.1696.

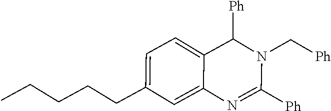 [SMILES: CCCCCc1ccc2c(c1)N=C(c1ccccc1)N(Cc1ccccc1)C2c1ccccc1]

3-Benzyl-7-pentyl-2,4-diphenyl-3,4-dihydroquinazoline (4). 30 Prepared according to the general dihydroquinazoline protocol with S4 (0.268 g, 1.00 mmol), benzylamine (0.12 mL, 1.1 mmol), benzaldehyde (0.11 mL, 1.1 mmol), CH₂Cl₂ (10.0 mL), 2-chloropyridine (0.11 mL, 1.2 mmol), and Tf₂O (0.18 mL, 1.1 mmol). The reaction proceeded for 48 h at rt after addition of Tf₂O and was worked up as described above. The residue was passed through a silica plug (90:10:0.5 EtOAc:hexanes:TEA as eluent) and the concentrated filtrate was further purified by flash chromatography (10% EtOAc in hexanes as eluent) to afford the desired product (0.201 g, 45% yield) as an oil. ¹H NMR (400 MHz, CDCl₃) δ 7.56-7.46 (m, 2H), 7.43-7.24 (m, 11H), 7.23-7.13 (m, 3H), 6.76 (dd, J=7.8, 1.8 Hz, 1H), 6.62 (d, J=7.7 Hz, 1H), 5.39 (s, 1H), 4.69 (d, J=15.7 Hz, 1H), 3.97 (d, J=15.7 Hz, 1H), 2.53 (dd, J=8.7, 6.7 Hz, 2H), 1.65-1.53 (m, 2H), 1.39-1.27 (m, 4H), 0.87 (t, J=7.0 Hz, 3H); ¹³C NMR (101 MHz, CDCl₃) δ 157.8, 144.0, 143.0, 141.0, 136.8, 136.6, 129.2, 129.0, 128.8, 128.6, 128.1, 128.0, 127.7, 127.4, 127.0, 126.2, 125.2, 124.8, 122.1, 60.7, 53.2, 35.6, 31.5, 30.8, 22.5, 14.0; IR (neat): 3060, 29280, 1584, 1549, 1493, 1422, 1321 cm⁻¹; HRMS (ESI): m/z calcd for C₃₂H₃₃N₂ [M+H], 445.2644; found, 445.2648.

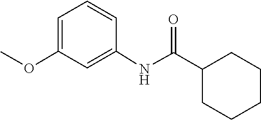 [SMILES: COc1cccc(NC(=O)C2CCCCC2)c1]

N-(3-methoxyphenyl)cyclohexanecarboxamide (S5). Prepared according to general amide protocol using m-anisidine (1.12 mL, 10 mmol), TEA (1.67 mL, 12 mmol), cyclohexanecarbonyl chloride (1.47 mL, 11 mmol), 4-DMAP (0.012 g, 0.098 mmol), and CH₂Cl₂ (50 mL). The reaction stirred overnight and was worked up as described above. The residual solid was purified by crystallization from EtOAc and hexanes to afford the desired product (1.635, 70% yield) as a solid (m.p.=94-96° C.). ¹H NMR (400 MHz, CDCl₃) δ 7.37 (s, 1H), 7.19 (t, J=8.1 Hz, 1H), 7.15 (s, 1H), 6.94 (dd, J=8.0, 2.1 Hz, 1H), 6.64 (dd, J=8.3, 2.5 Hz, 1H), 3.80 (s, 3H), 2.22 (tt, J=11.7, 3.5 Hz, 1H), 1.95 (dd, J=12.7, 3.4 Hz, 2H), 1.88-1.78 (m, 2H), 1.75-1.65 (m, 1H), 1.54 (qd, J=12.2, 10.7, 5.8 Hz, 2H), 1.38-1.17 (m, 3H); ¹³C NMR (101 MHz, CDCl₃) δ 174.4, 160.2, 139.4, 129.6, 111.6, 110.2, 105.2, 55.3, 46.7, 29.7, 25.7.

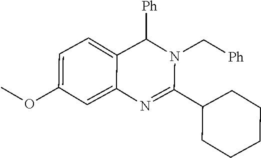 [SMILES: COc1ccc2c(c1)N=C(C1CCCCC1)N(Cc1ccccc1)C2c1ccccc1]

3-Benzyl-2-cyclohexyl-7-methoxy-4-phenyl-3,4-dihydroquinazoline (5). Prepared according to the general dihydroquinazoline protocol with S5 (0.233 g, 1.00 mmol), benzylamine (0.12 mL, 1.1 mmol), benzaldehyde (0.11 mL, 1.1 mmol), CH₂Cl₂ (10.0 mL), 2-chloropyridine (0.11 mL, 1.2 mmol), and Tf₂O (0.18 mL, 1.1 mmol). The reaction 30 proceeded for 24 h at rt after addition of Tf₂O and was worked up as described above. The residue was passed through a silica plug (90:10:0.5 EtOAc:hexanes:TEA as eluent) and the concentrated filtrate was further purified by flash chromatography (5% ether in CH₂Cl₂ as eluent) to afford the desired product (0.155 g, 38% yield) as a solid (m.p.=51-54° C.). ¹H NMR (400 MHz, CDCl₃) δ 7.40-7.19 (m, 10H), 6.83 (s, 1H), 6.58 (d, J=8.4 Hz, 1H), 6.47 (dd, J=8.4, 2.6 Hz, 1H), 5.29 (s, 1H), 4.82 (d, J=16.6 Hz, 1H), 4.09 (d, J=16.6 Hz, 1H), 3.77 (s, 3H), 2.60-2.38 (m, 1H), 2.01-1.55 (m, 7H), 1.42-1.07 (m, 3H); ¹³C NMR (101 MHz, CDCl₃) δ 162.3, 159.7, 144.2, 142.4, 136.8, 129.0, 128.9, 128.0, 127.7, 126.9, 126.7, 126.6, 116.9, 111.8, 107.9, 62.3, 55.3, 51.2, 41.0, 31.1, 30.2, 26.5, 26.1, 25.8; IR (neat): 3027, 2924, 1588, 1552, 1493, 1273, 1150, 1031 cm⁻¹; HRMS (ESI): m/z calcd for C₂₈H₃₁N₂O [M+H], 411.2436; found, 411.2437.

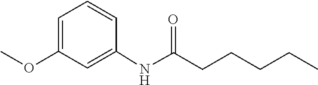 [SMILES: CCCCCC(=O)Nc1cccc(OC)c1]

N-(3-methoxyphenyl)hexanamide (S6). Prepared according to the general amide protocol using m-anisidine (1.12 mL, 9.97 mmol), TEA (1.67 mL, 12.0 mmol), hexanoyl chloride (1.54 mL, 11.0 mmol), 4-DMAP (0.012 g, 0.10 mmol), and CH₂Cl₂ (75 mL). The reaction stirred for 3 h and was worked up as described above. The residue was purified by flash chromatography (20% EtOAc in hexanes as eluent) to afford the desired product (1.982 g, 90% yield) as an oil. ¹H NMR (400 MHz, CDCl₃) δ 8.26 (s, 1H), 7.32 (t, J=2.3 Hz, 1H), 7.15 (t, J=8.1 Hz, 1H), 7.03 (d, J=8.0 Hz, 1H), 6.62 (dd, J=8.3, 2.5 Hz, 1H), 3.72 (s, 3H), 2.33 (t, J=7.6 Hz, 2H), 1.68 (p, J=7.6 Hz, 2H), 1.35-1.22 (m, 4H), 0.87 (t, J=7.0 Hz, 3H); ¹³C NMR (101 MHz, CDCl₃) δ 172.4, 160.0, 139.5, 129.5, 112.3, 109.9, 105.8, 55.2, 37.6, 31.4, 25.4, 22.4, 13.9; IR (neat): 3301, 2930, 1661, 1597, 1543, 1284, 1156, 1046 cm⁻¹; HRMS (ESI): m/z calcd for C₁₃H₂₀NO₂ [M+H], 222.1494; found, 222.1498.

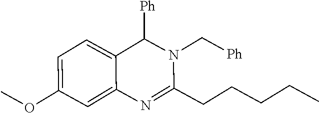 [SMILES: CCCCCC1=Nc2cc(OC)ccc2C(c2ccccc2)N1Cc1ccccc1]

3-Benzyl-2-hexyl-7-methoxy-4-phenyl-3,4-dihydroquinazoline (8). Prepared according to the general dihydroquinazoline protocol with S6 (0.221 g, 1.00 mmol), benzylamine (0.12 mL, 1.1 mmol), benzaldehyde (0.11 mL, 1.1 mmol), CH₂Cl₂ (10.0 mL), 2-chloropyridine (0.11 mL, 1.2 mmol), and Tf₂O (0.18 mL, 1.1 mmol). The reaction proceeded for 24 h at rt after addition of Tf₂O and was worked up as described above. The residue was passed through a silica plug (90:10:0.5 EtOAc:hexanes:TEA as eluent) and the concentrated filtrate was further purified by flash chromatography (50% EtOAc in hexanes as eluent) to afford the desired product (0.231 g, 58% yield) as an oil. ¹H NMR (400 MHz, CDCl₃) δ 7.40-7.21 (m, 10H), 6.76 (d, J=2.6 Hz, 1H), 6.57 (d, J=8.4 Hz, 1H), 6.46 (dd, J=8.3, 2.6 Hz, 1H), 5.29 (s, 1H), 4.75 (d, J=16.5 Hz, 1H), 4.07 (d, J=16.5 Hz, 1H), 3.77 (s, 3H), 2.62-2.42 (m, 2H), 1.81-1.61 (m, 2H), 1.44-1.23 (m, 4H), 0.87 (t, J=7.1 Hz, 3H); ¹³C NMR (101 MHz, CDCl₃) δ 159.7, 159.5, 144.1, 142.2, 136.4, 129.0, 128.9, 128.0, 127.8, 127.1, 126.9, 126.8, 117.0, 111.7, 107.8, 61.7, 55.3, 51.4, 35.5, 31.8, 27.2, 22.4, 14.0; IR (neat): 3029, 2926, 1590, 1556, 1495, 1452, 1275, 1150, 1034 cm⁻¹; HRMS (ESI): m/z calcd for C₂₇H₃₁N₂O [M+H], 399.2436; found, 399.2443.

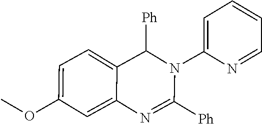 [SMILES: COc1ccc2c(c1)N=C(c1ccccc1)N(c1ccccn1)C2c1ccccc1]

7-Methoxy-2,4-diphenyl-3-(2-pyridyl)-3,4-dihydroquinazoline (11). Prepared according to the general dihydroquinazoline protocol with N-(3-methoxyphenyl)benzamide (0.228 g, 1.0 mmol), 2-aminopyridine (0.103 g, 1.09 mmol), benzaldehyde (0.11 mL, 1.1 mmol), CH₂Cl₂ (10.0 mL), 2-chloropyridine (0.11 mL, 1.2 mmol), and Tf₂O (0.18 mL, 1.1 mmol). The reaction proceeded for 24 h at rt after addition of Tf₂O and was worked up as described above. The residue was purified by MPLC (12-33% EtOAc in hexanes as eluent) to afford the desired product (0.128 g, 33% yield) as a solid (m.p.=155-156° C.). ¹H NMR (400 MHz, CDCl₃) δ 8.40 (ddd, J=5.0, 2.0, 0.9 Hz, 1H), 7.64-7.59 (m, 2H), 7.52-7.46 (m, 2H), 7.34-7.17 (m, 7H), 7.12 (d, J=2.6 Hz, 1H), 7.09 (d, J=8.3 Hz, 1H), 6.86-6.79 (m, 2H), 6.77 (s, 1H), 6.38 (d, J=8.3 Hz, 1H), 3.88 (s, 3H); ¹³C NMR (101 MHz, CDCl₃) δ 159.8, 155.6, 153.8, 148.2, 143.2, 142.4, 136.4, 136.2, 130.2, 129.1, 128.44, 128.41, 127.5, 127.3, 127.0, 119.8, 117.9, 117.5, 113.1, 109.2, 59.0, 55.5; IR (neat): 3058, 2924, 1584, 1545, 1465, 1433, 1269, 1124, 1031 cm⁻¹; HRMS (ESI): m/z calcd for C₂₆H₂₂N₃O [M+H], 392.1763; found, 392.1766.

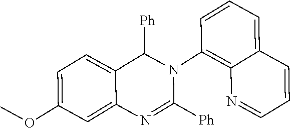 [SMILES: COc1ccc2c(c1)N=C(c1ccccc1)N(c1cccc3cccnc13)C2c1ccccc1]

7-Methoxy-2,4-diphenyl-3-(8-quinolyl)-3,4-dihydroquinazoline (13). Prepared according to the general dihydroquinazoline protocol with N-(3-methoxyphenyl)benzamide (0.227 g, 1.00 mmol), 8-aminoquinoline (0.159 g, 1.1 mmol), benzaldehyde (0.11 mL, 1.1 mmol), CH₂Cl₂ (10.0 mL), 2-chloropyridine (0.11 mL, 1.2 mmol), and Tf₂O (0.18 mL, 1.1 mmol). The reaction proceeded for 24 h at rt after addition of Tf₂O and was worked up as described above. The residue was passed through a silica plug (90:10:0.5 EtOAc:hexanes:TEA as eluent) and the concentrated filtrate was further purified by flash chromatography (5% ether in CH₂Cl₂ as eluent) to afford the desired product (0.144 g, 33% yield) as a solid (m.p.=172-174° C.). ¹H NMR (400 MHz, CDCl₃) δ 8.81 (dd, J=4.2, 1.7 Hz, 1H), 7.94 (dd, J=8.3, 1.8 Hz, 1H), 7.57-7.43 (m, 5H), 7.35-6.94 (m, 10H), 6.80 (d, J=8.3 Hz, 1H), 6.62 (dd, J=8.4, 2.6 Hz, 1H), 6.05 (s, 1H), 3.82 (s, 3H); ¹³C NMR (101 MHz, CDCl₃) δ 159.6, 157.9, 149.8, 145.4, 143.5, 142.6, 142.4, 137.7, 135.7, 128.8, 128.74, 128.70, 127.6, 127.4, 127.3, 126.9, 126.2, 125.8, 121.4, 118.9, 112.5, 109.0, 66.3, 55.3; IR (neat): 3058, 2928, 1588, 1545, 1489, 1385, 1273, 1113, 1031 cm⁻¹; HRMS (ESI): m/z calcd for C₃₀H₂₄N₃O [M+H], 442.1919; found, 442.1920.

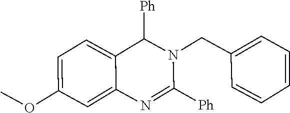 [SMILES: COc1ccc2c(c1)N=C(c1ccccc1)N(Cc1ccccc1)C2c1ccccc1]

7-Methoxy-2,4-diphenyl-3-[(2-pyridyl)methyl]-3,4-dihydroquinazoline (14). Prepared according to the general dihydroquinazoline protocol with N-(3-methoxyphenyl)benzamide (0.228 g, 1.00 mmol), 2-(aminomethyl)pyridine (0.11 g, 1.1 mmol), benzaldehyde (0.11 mL, 1.1 mmol), CH₂Cl₂ (10.0 mL), 2-chloropyridine (0.11 mL, 1.2 mmol), and Tf₂O (0.18 mL, 1.1 mmol). The reaction proceeded for 24 h at rt after addition of Tf₂O and was worked up as described above. The residue was purified by MPLC (60%-90% EtOAc in hexanes as eluent) to afford the desired product (0.113 g, 28% yield) as a dark yellow oil. ¹H NMR (400 MHz, CDCl₃) δ 8.53 (ddd, J=4.8, 1.8, 0.9 Hz, 1H), 7.62 (td, J=7.7, 1.8 Hz, 1H), 7.52-7.46 (m, 2H), 7.41-7.27 (m, 8H), 7.22 (d, J=7.9 Hz, 1H), 7.17 (ddd, J=7.7, 4.8, 1.1 Hz, 1H), 6.90 (d, J=2.6 Hz, 1H), 6.66 (dd, J=8.4, 0.6 Hz, 1H), 6.55 (dd, J=8.4, 2.6 Hz, 1H), 5.47 (s, 1H), 4.73 (d, J=16.4 Hz, 1H), 4.26 (d, J=16.4 Hz, 1H), 3.78 (s, 3H); ¹³C NMR (101 MHz, CDCl₃) δ 159.7, 158.2, 157.2, 149.7, 143.7, 142.4, 137.0, 136.3, 129.4, 129.0, 128.7, 128.1, 128.0, 127.2, 127.1, 122.6, 121.0, 117.2, 112.4, 108.7, 61.6, 55.29, 55.26; IR (neat): 3060, 2919, 1590, 1549, 1491, 1444, 1262, 1157, 1034 cm⁻¹; HRMS (ESI): m/z calcd for C₂₇H₂₄N₃O [M+H], 406.1919; found, 406.1924.

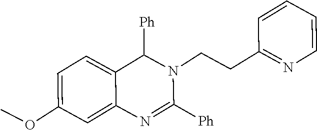 [SMILES: COc1ccc2c(c1)N=C(c1ccccc1)N(CCc1ccccn1)C2c1ccccc1]

7-Methoxy-2,4-diphenyl-3-[2-(2-pyridyl)ethyl]-3,4-dihydroquinazoline (15). Prepared according to the general dihydroquinazoline protocol with N-(3-methoxyphenyl)benzamide (0.227 g, 1.00 mmol), 2-(2-pyridyl)ethylamine (0.13 mL, 1.1 mmol), benzaldehyde (0.11 mL, 1.1 mmol), CH₂Cl₂ (10.0 mL), 2-chloropyridine (0.11 mL, 1.2 mmol), and Tf₂O (0.18 mL, 1.1 mmol). The reaction proceeded for 24 h at rt after addition of Tf₂O and was worked up as described above. The residue was passed through a silica plug (90:10:0.5 EtOAc:hexanes:TEA as eluent) and the concentrated filtrate was further purified by flash chromatography (70% EtOAc in hexanes as eluent) to afford the desired product (0.119 g, 29% yield) as an oil. ¹H NMR (400 MHz, CDCl₃) δ 8.40 (ddd, J=4.9, 1.9, 0.9 Hz, 1H), 7.45-7.21 (m, 11H), 7.05 (ddd, J=7.6, 4.9, 1.2 Hz, 1H), 6.82 (d, J=2.6 Hz, 1H), 6.75 (dd, J=9.3, 8.1 Hz, 2H), 6.58 (dd, J=8.4, 2.6 Hz, 1H), 5.48 (s, 1H), 3.82 (dt, J=14.6, 7.3 Hz, 1H), 3.77 (s, 3H), 3.47 (dt, J=14.5, 6.5 Hz, 1H), 2.92 (t, J=7.1 Hz, 2H); ¹³C NMR (101 MHz, CDCl₃) δ 159.7, 158.2, 149.4, 144.7, 142.8, 136.5, 136.4, 129.4, 129.0, 128.5, 128.1, 128.0, 126.8, 126.5, 123.5, 121.6, 117.5, 112.2, 108.5, 61.9, 55.3, 50.8, 37.5; IR (neat): 3025, 2935, 1586, 1545, 1489, 1275, 1146, 1034 cm⁻¹; HRMS (ESI): m/z calcd for C₂₈H₂₆N₃O [M+H], 420.2076; found, 420.2067.

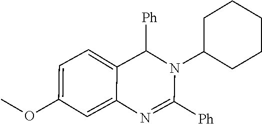 [SMILES: COc1ccc2c(c1)N=C(c1ccccc1)N(C1CCCCC1)C2c1ccccc1]

3-Cyclohexyl-7-methoxy-2,4-diphenyl-3,4-dihydroquinazoline (16). Prepared according to the general dihydroquinazoline protocol with N-(3-methoxyphenyl)benzamide (0.228 g, 1.00 mmol), cyclohexylamine (0.13 mL, 1.1 mmol), benzaldehyde (0.11 mL, 1.1 mmol), CH₂Cl₂ (10.0 mL), 2-chloropyridine (0.11 mL, 1.2 mmol), and Tf₂O (0.18 mL, 1.1 mmol). The reaction proceeded for 48 h at rt after addition of Tf₂O and was worked up as described above. The residue was passed through a silica plug (90:10:0.5 EtOAc:hexanes:TEA as eluent) and the concentrated filtrate was further purified by flash chromatography (70% EtOAc in hexanes as eluent) to afford the desired product (0.095 g, 24% yield) as a solid (m.p.=77-80° C.). ¹H NMR (400 MHz, CDCl₃) δ 7.67-7.59 (m, 2H), 7.42-7.28 (m, 5H), 7.22-7.15 (m, 2H), 7.14-7.07 (m, 1H), 6.89 (d, J=8.3 Hz, 1H), 6.81 (s, 1H), 6.57 (dd, J=8.3, 2.6 Hz, 1H), 5.51 (s, 1H), 3.70 (s, 3H), 3.53 (ddd, J=11.7, 8.0, 5.0 Hz, 1H), 1.75-1.65 (m, 1H), 1.66-1.52 (m, 3H), 1.51-1.31 (m, 3H), 1.03-0.86 (m, 3H); ¹³C NMR (101 MHz, CDCl₃) δ 159.7, 159.3, 146.4, 143.1, 137.1, 130.0, 128.8, 128.6, 128.4, 127.3, 126.0, 125.5, 119.1, 112.3, 108.4, 60.9, 56.7, 55.3, 33.1, 31.9, 25.9, 25.7, 25.2; IR (neat): 3060, 2932, 1584, 1538, 1489, 1275, 1142, 1034 cm⁻¹; HRMS (ESI): m/z calcd for C₂₇H₂₉N₂O [M+H], 397.228; found, 397.2279.

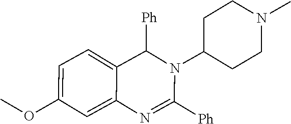

7-Methoxy-3-(1-methyl-4-piperidyl)-2,4-diphenyl-3,4-dihydroquinazoline (17). Prepared according to the general dihydroquinazoline protocol with N-(3-methoxyphenyl)benzamide (0.230 g, 1.01 mmol), 4-amino-1-methylpiperidine (0.14 mL, 1.1 mmol), benzaldehyde (0.11 mL, 1.1 mmol), CH₂Cl₂ (10.0 mL), 2-chloropyridine (0.11 mL, 1.2 mmol), and Tf₂O (0.18 mL, 1.1 mmol). The reaction proceeded for 24 h at rt after addition of Tf₂O and was worked up as described above. The residue was passed through a silica plug (90:10:0.5 EtOAc:hexanes:TEA as eluent) and the concentrated filtrate was further purified by flash chromatography (17:80:3 ether:CH₂Cl₂:MeOH as eluent) to afford the desired product (0.162 g, 39% yield) as a solid (m.p.=125-128° C.). ¹H NMR (400 MHz, CDCl₃) δ 7.70 (dd, J=7.6, 2.0 Hz, 2H), 7.51-7.36 (m, 5H), 7.30-7.23 (m, 2H), 7.22-7.16 (m, 1H), 6.95 (d, J=8.3 Hz, 1H), 6.89 (d, J=2.6 Hz, 1H), 6.65 (dd, J=8.3, 2.6 Hz, 1H), 5.65 (s, 1H), 3.78 (s, 3H), 3.72-3.57 (m, 1H), 2.88-2.79 (m, 2H), 2.22 (s, 3H), 2.18-1.93 (m, 2H), 1.81 (qd, J=12.1, 2.6 Hz, 2H), 1.70 (ddd, J=12.7, 4.2, 2.3 Hz, 1H), 1.48 (ddd, J=12.9, 4.5, 2.2 Hz, 1H); ¹³C NMR (101 MHz, CDCl₃) δ 159.6, 159.1, 146.2, 142.9, 136.9, 130.1, 128.8, 128.7, 128.3, 127.4, 126.0, 125.4, 119.1, 112.4, 108.6, 58.5, 56.6, 55.3, 55.1, 54.9, 45.8, 31.8, 30.7; IR (neat): 3058, 2939, 1539, 1489, 1277, 1128, 1033 cm⁻¹; HRMS (ESI): m/z calcd for C₂₇H₃₉N₃O [M+H], 412.2389; found, 412.2386.

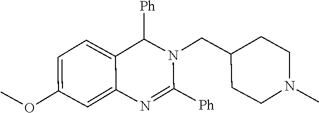

7-Methoxy-3-[(1-methyl-4-piperidyl)methyl]-2,4-diphenyl-3,4-dihydroquinazoline (19). Prepared according to the general dihydroquinazoline protocol with N-(3-methoxyphenyl)benzamide (0.228 g, 1.00 mmol), (1-Methyl-4-piperidinyl)methanamine (0.142 g, 1.11 mmol), benzaldehyde (0.11 mL, 1.1 mmol), CH₂Cl₂ (10.0 mL), 2-chloropyridine (0.22 mL, 2.4 mmol), and Tf₂O (0.18 mL, 1.1 mmol). The reaction proceeded for 24 h at rt after addition of Tf₂O and was worked up as described above. The residue was purified by MPLC (5-30% MeOH in CH₂Cl₂ as eluent) to afford the desired product (0.082 g, 19% yield) as a solid (m.p.=68-71° C.). ¹H NMR (400 MHz, CDCl₃) δ 7.49-7.22 (m, 10H), 6.86 (d, J=2.6 Hz, 1H), 6.81 (d, J=8.3 Hz, 1H), 6.60 (dd, J=8.4, 2.6 Hz, 1H), 5.48 (s, 1H), 3.78 (s, 3H), 3.28 (dd, J=14.4, 9.3 Hz, 1H), 2.97 (dd, J=14.3, 5.3 Hz, 1H), 2.82-2.70 (m, 2H), 2.22 (s, 3H), 1.89 (td, J=11.6, 2.4 Hz, 1H), 1.82 (td, J=11.8, 2.6 Hz, 1H), 1.75-1.62 (m, 1H), 1.62-1.48 (m, 2H), 1.12 (qd, J=12.1, 4.0 Hz, 1H), 1.00 (qd, J=12.1, 4.0 Hz, 1H); ¹³C NMR (101 MHz, CDCl₃) δ 159.7, 158.5, 144.2, 142.7, 136.4, 129.3, 129.0, 128.5, 128.3, 128.1, 126.9, 126.5, 117.3, 112.2, 108.5, 61.6, 55.5, 55.3, 55.2, 55.1, 46.0, 33.6, 29.7, 29.3; IR (neat): 3025, 2930, 1582, 1541, 1489, 1275, 1142, 1031 cm⁻¹; HRMS (ESI): m/z calcd for C₂₈H₃₂N₃O [M+H], 426.2545; found, 426.2548.

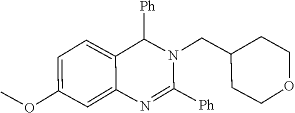 [SMILES: COc1ccc2c(c1)N=C(c1ccccc1)N(CC1CCOCC1)C2c1ccccc1]

7-Methoxy-2,4-diphenyl-3-[(tetrahydro-2H-pyran-4-yl)methyl]-3,4-dihydroquinazoline (20). Prepared according to the general dihydroquinazoline protocol with N-(3-methoxyphenyl)benzamide (0.223 g, 0.981 mmol), 4-aminomethyltetrahydropyran (0.12 mL, 1.1 mmol), benzaldehyde (0.11 mL, 1.1 mmol), CH₂Cl₂ (10.0 mL), 2-chloropyridine (0.11 mL, 1.2 mmol), and Tf₂O (0.18 mL, 1.1 mmol). The reaction proceeded for 24 h at rt after addition of Tf₂O and was worked up as described above. The residue was passed through a silica plug (90:10:0.5 EtOAc:hexanes:TEA as eluent) and the concentrated filtrate was further purified by flash chromatography (20:80:1 ether:CH₂Cl₂:MeOH as eluent) to afford the desired product (0.105 g, 26% yield) as an oil. ¹H NMR (400 MHz, CDCl₃) δ 7.47-7.26 (m, 10H), 6.88 (d, J=2.6 Hz, 1H), 6.82 (d, J=8.4 Hz, 1H), 6.61 (dd, J=8.4, 2.6 Hz, 1H), 5.49 (s, 1H), 3.90-3.80 (m, 2H), 3.78 (s, 3H), 3.37-3.19 (m, 3H), 2.99 (dd, J=14.4, 5.3 Hz, 1H), 2.01-1.86 (m, 1H), 1.53-1.43 (m, 2H), 1.18-0.90 (m, 2H); ¹³C NMR (101 MHz, CDCl₃) δ 159.8, 158.4, 144.1, 142.5, 136.3, 129.5, 129.1, 128.6, 128.4, 128.1, 126.9, 126.5, 117.2, 112.3, 108.5, 67.5, 67.4, 61.7, 55.8, 55.3, 33.7, 30.5, 30.2; IR (neat): 3064, 2932, 1586, 1543, 1491, 1273, 1142, 1094, 1034 cm⁻¹; HRMS (ESI): m/z calcd for C₂₇H₂₉N₂O₂ [M+H], 413.2229; found, 413.2226.

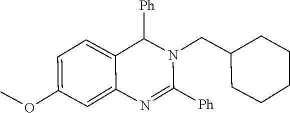 [SMILES: COc1ccc2c(c1)N=C(c1ccccc1)N(CC1CCCCC1)C2c1ccccc1]

3-(Cyclohexylmethyl)-7-methoxy-2,4-diphenyl-3,4-dihydroquinazoline (21). Prepared according to the general dihydroquinazoline protocol with N-(3-methoxyphenyl)benzamide (0.229 g, 1.01 mmol), cyclohexanemethylamine (0.14 mL, 1.1 mmol), benzaldehyde (0.11 mL, 1.1 15 mmol), CH₂Cl₂ (10.0 mL), 2-chloropyridine (0.11 mL, 1.2 mmol), and Tf₂O (0.18 mL, 1.1 mmol). The reaction proceeded for 24 h at rt after addition of Tf₂O and was worked up as described above. The residue was passed through a silica plug (90:10:0.5 EtOAc:hexanes:TEA as eluent) and the concentrated filtrate was further purified by flash chromatography (70% EtOAc in hexanes as eluent) to afford the desired product (0.188 g, 46% yield) as an oil. ¹H NMR (400 MHz, CDCl₃) δ 7.45-7.23 (m, 10H), 6.86 (d, J=2.6 Hz, 1H), 6.80 (d, J=8.3 Hz, 1H), 6.59 (dd, J=8.4, 2.6 Hz, 1H), 5.50 (s, 1H), 3.77 (s, 3H), 3.20 (dd, J=14.2, 9.4 Hz, 1H), 2.90 (dd, J=14.2, 4.9 Hz, 1H), 1.75-1.47 (m, 6H), 1.23-0.94 (m, 3H), 0.79-0.48 (m, 2H); ¹³C NMR (101 MHz, CDCl₃) δ 159.7, 158.6, 144.5, 142.9, 136.9, 129.2, 129.0, 128.5, 128.4, 128.0, 127.0, 126.6, 117.4, 112.0, 108.5, 61.4, 56.3, 55.3, 36.2, 30.9, 30.4, 26.3, 25.9, 25.7; IR (neat): 3062, 2924, 1586, 1541, 1491, 1446, 1273, 1146, 1034 cm⁻¹; HRMS (ESI): m/z calcd for C₂₈H₃₁N₂O [M+H], 411.2436; found, 411.2440.

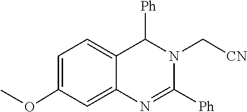 [SMILES: COc1ccc2c(c1)N=C(c1ccccc1)N(CC#N)C2c1ccccc1]

(7-Methoxy-2,4-diphenyl-3,4-dihydroquinazolin-3-yl)acetonitrile (22). Prepared according to the general dihydroquinazoline protocol with N-(3-methoxyphenyl)benzamide (0.227 g, 1.00 mol), aminoacetonitrile HCl (0.102 g, 1.1 mmol), benzaldehyde (0.11 mL, 1.1 mmol), CH₂Cl₂ (10.0 mL), Tf₂O (0.18 mL, 1.1 mmol), and 2-chloropyridine (0.22 mL, 2.4 mmol), with half of the 2-chloropyridine (0.11 mL) added to the initial amide, amine HCl salt, and aldehyde mixture. The reaction proceeded for 24 h at rt after addition of Tf₂O and was worked up as described above. The residue was passed through a silica plug (90:10:0.5 EtOAc:hexanes:TEA as eluent) and the concentrated filtrate was further purified by flash chromatography (5% ether in CH₂Cl₂ as eluent) to afford the desired product (0.180 g, 51% yield) as a solid (m.p.=138-140° C.). ¹H NMR (400 MHz, CDCl₃) δ 7.52-7.30 (m, 10H), 6.87 (d, J=2.6 Hz, 1H), 6.74 (dd, J=8.4, 0.7 Hz, 1H), 6.63 (dd, J=8.4, 2.6 Hz, 1H), 5.73 (s, 1H), 4.14 (d, J=17.8 Hz, 1H), 3.80 (d, J=17.9, 1H), 3.77 (s, 3H); ¹³C NMR (101 MHz, CDCl₃) δ 160.0, 155.9, 142.1, 141.1, 135.0, 130.2, 129.4, 129.2, 128.9, 127.8, 127.5, 127.3, 116.8, 114.9, 113.3, 109.5, 63.0, 55.4, 39.3; IR (neat): 3029, 2928, 1558, 1491, 1429, 1264, 1163, 1032 cm⁻¹; HRMS (ESI): m/z calcd for C₂₃H₂₀N₃O [M+H], 354.1606; found, 354.1606.

Screening of this library of dihydroquinazolines was performed in two stages. In the first stage, each compound was screened at three concentrations (3, 10 and 30 μM) to select lead agents. Secondly, lead agents were further analyzed using full concentration responses (6-point titration ranging from 1.25 μM-40 μM) for each of the three proteolytic activities of the 20S proteasome and a combination thereof. The proteolytic activity of the 20S proteasome can be monitored in vitro by measuring the cleavage of fluorogenic peptide substrates for the different catalytic sites as an increase in 7-amino-4-methycoumarin (AMC) fluorescence overtime. A combination of chymotrypsin-like (CT-L, Suc-LLVY-AMC), tryptic-like (T-L, Boc-LRR-AMC) and caspase-like (Casp-L, Z-LLE-AMC) peptide substrates were used in equal amounts to screen compounds for overall 20S activity.

Pure human 20S proteasome was pre-treated with 3, 10 or 30 μM of one of the analogues or DMSO (vehicle control) for 15 min at 37° C. To each sample was then added a mixture of the three substrates (13.3 μM each). The release of AMC was monitored as fluorescence overtime for 1 h and the resulting 20S activity changes were determined by comparing to the untreated 20S and calculating the fold-increase in activity for each analogue at a given concentration (Table 1, 30 μM).

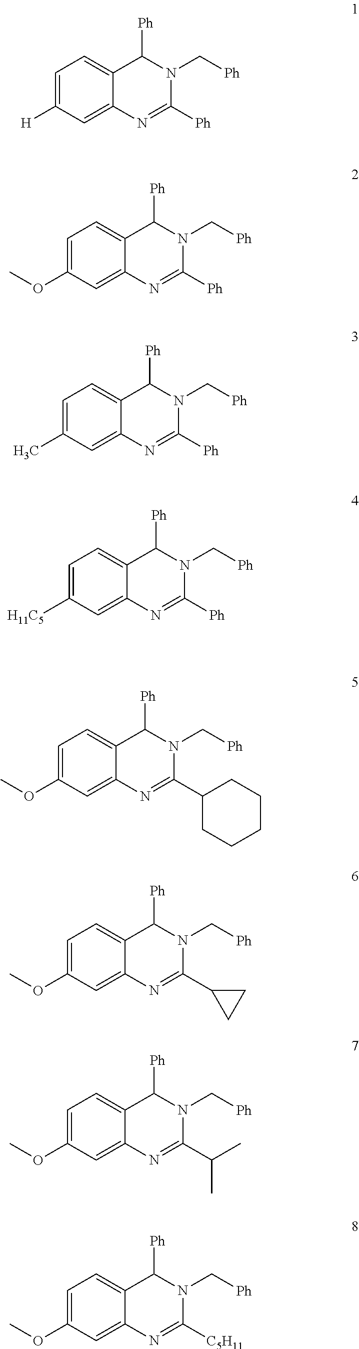

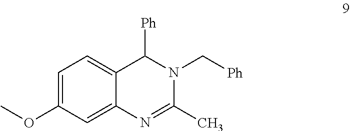 [SMILES: COc1ccc2c(c1)N=C(C)N(Cc1ccccc1)C2c1ccccc1]

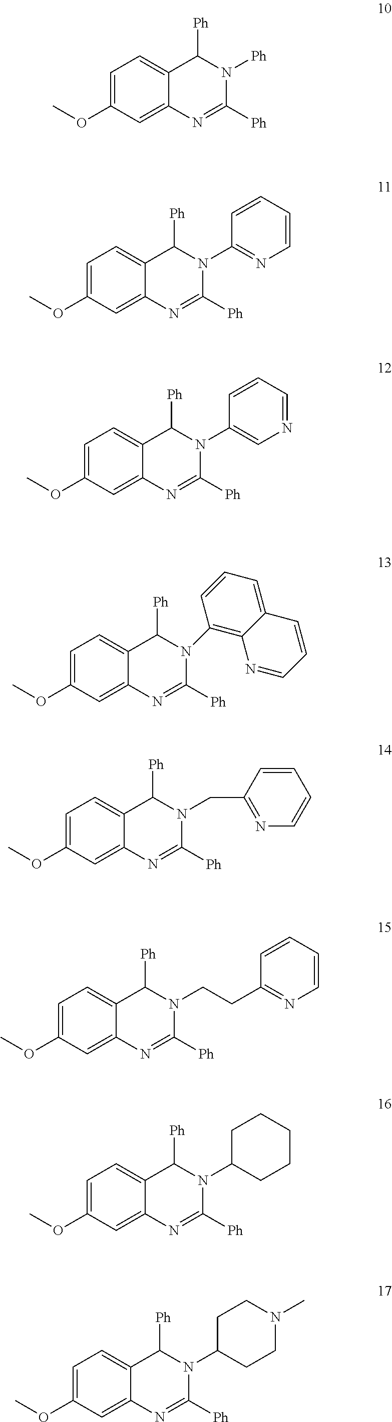

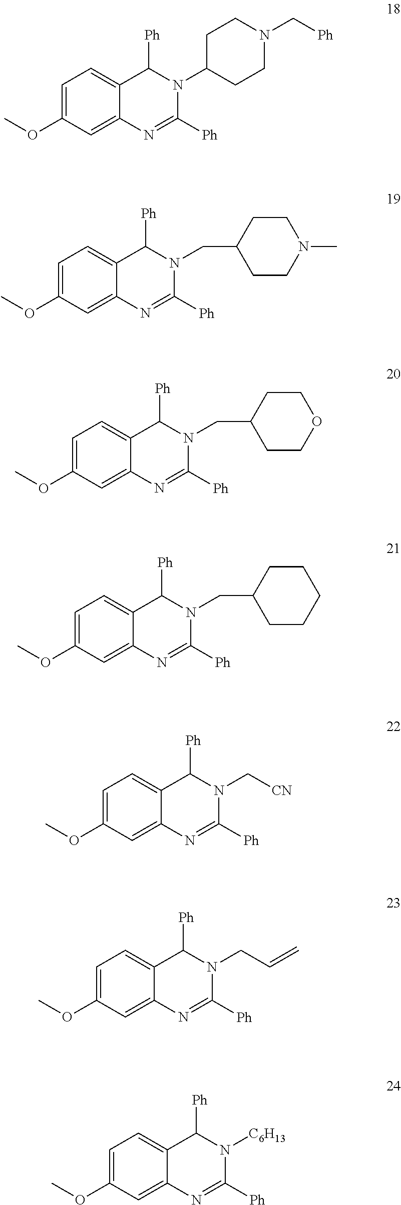

TABLE 1
Compounds 1-24 ranked by fold enhancement
of 20S activity at 30 μM

| Compound | Fold increase over vehicle (30 μM) |
|---|---|
| 10 | 9.5 |
| 21 | 8.2 |
| 3 | 8.1 |
| 2 | 7.9 |
| 18 | 7 |
| 11 | 6.9 |
| 16 | 6.7 |
| 5 | 6.5 |
| 12 | 5.9 |
| 13 | 5.3 |
| 1 | 4 |
| 8 | 2.8 |
| 15 | 2.8 |
| 4 | 2.8 |
| 6 | 2.7 |
| 22 | 2.7 |
| 7 | 2.3 |
| 23 | 2.3 |
| 14 | 2 |
| 9 | 1.8 |
| 24 | 1.6 |
| 20 | 1.6 |
| 19 | 1.2 |
| 17 | 1 |

The data collected from this screen (Table 1) shows a few insightful trends in the SAR of the dihydroquinazolines. Small changes in substitution at the 7-position appear to have a significant effect on activity of the dihydroquinazolines. Compound 1, which displays a 4-fold (i.e. 400%) increase over background 20S activity, lacks a substituent at the 7-position but is otherwise identical to compounds 2 (7.9-fold increase) and 3 (8.1-fold increase). This small change results in a reduction in 20S activity from 8-fold enhancement down to a 4-fold enhancement at 30 μM Similarly, the addition of a longer alkyl chain on compound 4 resulted in a steep drop in 20S activity to 2.8-fold at 30 μM. Changes at the 2-position show similar effects to that of the 7-position, where most substitutions other than a phenyl group (compounds 5-9) caused marked decreases in 20S activity. All have <3-fold activation at 30 μM, apart from compound 5 (6.5-fold increase).

Substitutions at the 3-position showed more flexibility to changes than either the 7- or 2-positions, while still having a significant effect on the relative 20S activities of the analogues. Many of the most potent analogues, like compounds 10, 3, 2 and 5 (9.5, 8.1, 7.9 and 6.5-fold increase of 20S activity, respectively), contain a phenyl or benzyl functionality at the 3-position. Other similarly sized and shaped substituents like cyclohexane (compound 16 (6.7-fold)) and pyridine compounds 11 (6.9-fold) and 12 (5.9-fold)) also provided some of the most potent analogues. Interestingly, larger substituents at the 3-position as seen in compounds 18 (7-fold) and 13 (5.3-fold) also yielded potent analogues, suggesting that additional functionalities may be incorporated here for further optimization if necessary. The substitution of the phenyl or benzyl groups for some other heterocycles such as N-methylpiperidine (compounds 17 (1-told) and 19 (1.2-fold)), tetrahydropyran (compound 20 (1.6-fold)) or even a pyridine linked by a methylene group in compound 14 (2-fold) lead to significant decreases in 20S activity. This suggests that placement of heteroatoms at the 3-position may disrupt hydrophobic interactions in that region. The difference in activity shown between 17 and 18 could be caused by a disruption of hydrophobic interactions with the addition of the piperidine nitrogen, which could then be replaced by new interactions made by the phenyl group in 18. The addition of non-cyclic substituents at the 3-position (compounds 22, 23 and 24) resulted in very little 20S activity (2.7, 2.3 and 1.6-fold increase in 20S activity, respectively) in all cases suggesting that larger hydrophobic groups at the 3-position are likely required for 20S activity.

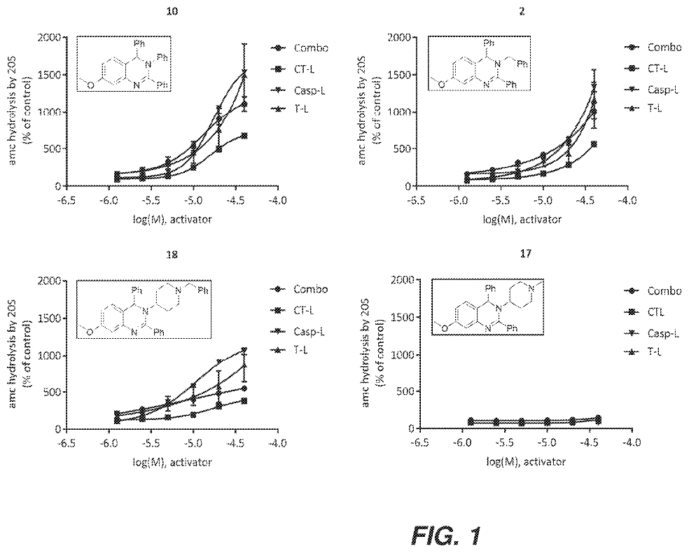

After analyzing the results in Table 1, three of the most promising analogues were selected for further studies into their 20S activity. Compounds 10, 2 and 18 were selected to be carried forward based on their fold increase and the highest Max-Fold activities. Compound 17 was also carried forward to use as a negative control since it had no discemible activity towards the 20S. These compounds were then tested to obtain a full concentration—response (FIG. 1) of their activities towards the 20S proteasome using each of the three substrates for the three catalytic sites individually and the combination of the three substrates. This was done to ensure that each of the selected compounds activate the 20S proteasome at all three catalytic sites, which is critical for effective IDP degradation, as these proteins are likely to contain multiple cleavage sites for each. Previously identified 20S proteasome activators that were only able to activate a single catalytic site showed poor enhancement of IDP degradation in vitro when compared to those that activated all three catalytic sites.

Using the data in FIG. 1, the concentration at which 20S activity was doubled (AC₂₀₀) was calculated for each compound using each substrate and the combination of the three substrates (Table 2).

TABLE 2
Detailed Analysis of 20S activation by select dihydroquinazoline analogs

| | Combo | | CT-L | | Casp-L | | T-L | |
|---|---|---|---|---|---|---|---|---|
| Compound | AC₂₀₀ (μM) | Max Fold | AC₂₀₀ (μM) | Max Fold | AC₂₀₀ (μM) | Max Fold | AC₂₀₀ (μM) | Max Fold |
| 10 | | 11.1 | 8.1 | 6.7 | 5.8 | 15.3 | 2.5 | 15.0 |
| 2 | 2.0 | 10.1 | 12.9 | 5.6 | 5.6 | 13.4 | 5.0 | 11.7 |
| 18 | 1.3 | 5.5 | 10.5 | 3.8 | 2.8 | 10.6 | 1.7 | 8.7 |
| 17 | N/A | 1.4 | N/A | 1.1 | N/A | 0.8 | N/A | 1.0 |

Because of variations in the maximum fold enhancement between 20S enhancers, AC₂₀₀ values allow for easy comparisons to be made between activators. It was found that each of the active compounds (10, 2 and 18) achieved both high maximum fold increases (>500%) in 20S activity and doubled 20S activity in the combination at low μM concentrations.

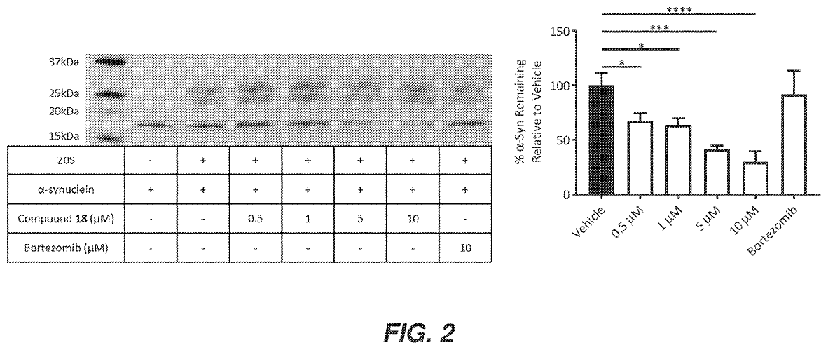

Although the three compounds showed near equipotent activities, compound 18 was selected to move forward with due to its lowest overall AC₂₀₀ values (Table 2: combo, AC₂₀₀ 1.3 μM) and the individual activities of the catalytic sites of the 20S. The efficacy of compound 18 was tested by observing its ability to enhance 20S-mediated degradation of α-synuclein, the IDP associated with the development of Parkinson's Disease. Briefly, the 20S proteasome was incubated with compound 18, followed by addition of pure human α-synuclein. This mixture was then incubated for 4 h at 37° c. The digestions were analyzed using silver stain and quantified (FIG. 2 to determine the ability of compound 18 to enhance IDP degradation by the 20S. It was found that compound 18 effectively enhanced the rate of degradation of α-synuclein by the 20S in vitro in a concentration dependent manner. As a control, we used the proteasome inhibitor, bortezomib, which prevented α-synuclein degradation, confirming that the clearance of α-synuclein in FIG. 2 is a proteasome mediated event This shows that the prior peptide substrate-based results are likely to translate to the degradation of full IDPs and that dihydroquinazolines represent a promising new lead from which potent 20S activators that enhance IDP degradation can be developed.

In conclusion, this study demonstrates that dihydroquinazolines represent a promising scaffold from which potent 20S activators can be developed. Additionally, recently developed synthetic methods allow for access to a broad scope of dihydroquinazoline analogues, allowing for exploration of a variety of different substituents and substitution patterns. Among the analogues tested, we found several active compounds and a few of the most potent 20S activators identified to date.

Additional compounds that are described herein include the following.

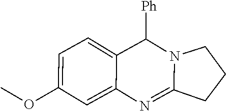 [SMILES: COc1ccc2c(c1)N=C1CCCN1C2c1ccccc1]

Prepared according to the general dihydroquinazoline protocol with 4-amino-N-(3-methoxyphenyl)butanamide (0.104 g, 0.50 mmol), benzaldehyde (0.060 mL, 0.55 mmol), CH₂Cl₂ (25 mL), 2-chloropyridine (0.060 mL, 0.60 mmol), and Tf₂O (0.090 mL, 0.55 mmol). The reaction proceeded for 24 h at rt after addition of Tf₂O and was worked up as described above. The residue was purified by MPLC (63-84% EtOAc in hexanes as eluent on an amino functionalized silica gel column) to afford the desired product (0.111 g, 80% yield) as a solid (m.p.=140-144° C.). ¹H NMR (400 MHz, CDCl₃) δ 7.37-7.16 (m, 5H), 6.69 (d, J=2.6 Hz, 1H), 6.57 (dd, J=8.4, 0.7 Hz, 1H), 6.45 (dd, J=8.4, 2.6 Hz, 1H), 5.54 (s, 1H), 3.74 (s, 3H), 3.10 (ddd, J=9.7, 8.0, 5.9 Hz, 2H), 2.69 (ddd, J=8.5, 7.2, 2.4 Hz, 2H), 2.04-1.83 (m, 2H); ¹³C NMR (101 MHz, CDCl₃) δ 161.7, 159.6, 143.6, 142.9, 128.7, 127.9, 127.8, 127.4, 115.6, 110.7, 108.0, 61.3, 55.1, 49.3, 31.8, 18.8; IR (neat): 2947, 1597, 1567, 1493, 1286, 1159 cm⁻¹; HRMS (ESI): m/z calcd for C₁₈H₁₉N₂O [M+H], 279.1497; found, 279.1497.

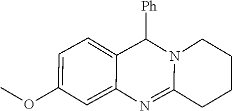 [SMILES: COc1ccc2c(c1)N=C1CCCCN1C2c1ccccc1]

Prepared according to the general dihydroquinazoline protocol with 5-amino-N-(3-methoxyphenyl)pentanamide (0.222 g, 1.0 mmol), benzaldehyde (0.11 mL, 1.1 mmol), CH₂Cl₂ (10 mL), 2-chloropyridine (0.11 mL, 1.2 mmol), and Tf₂O (0.19 mL, 1.1 mmol). The reaction proceeded for 24 h at rt after addition of Tf₂O and was worked up as described above. The residue was purified by MPLC (65-85% EtOAc in hexanes as eluent on an amino functionalized silica gel column) to afford the desired product (0.140 g, 48% yield) as a solid (m.p.=159-161° C.). ¹H NMR (400 MHz, CDCl₃) δ 7.34-7.16 (m, 5H), 6.66 (d, J=2.6 Hz, 1H), 6.62 (dd, J=8.3, 0.7 Hz, 1H), 6.45 (dd, J=8.4, 2.6 Hz, 1H), 5.28 (s, 1H), 3.73 (s, 3H), 3.02 (t, J=6.2 Hz, 2H), 2.75-2.64 (m, 1H), 2.63-2.51 (m, 1H), 1.84-1.69 (m, 3H), 1.69-1.55 (m, 1H); ¹³C NMR (101 MHz, CDCl₃) δ 159.6, 156.2, 144.1, 142.3, 128.8, 127.8, 127.0, 126.7, 116.7, 110.9, 107.2, 64.9, 55.1, 48.2, 32.1, 23.3, 20.3; IR (neat): 2945, 1590, 1552, 1493, 1292, 1150 cm⁻¹; HRMS (ESI): m/z calcd for C₁₉H₂₁N₂O [M+H], 293.1654; found, 293.1654.

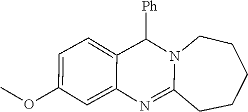 [SMILES: COc1ccc2c(c1)N=C1CCCCCN1C2c1ccccc1]

Prepared according to the general dihydroquinazoline protocol with 6-amino-N-(3-methoxyphenyl)hexanamide (0.236 g, 1.0 mmol), benzaldehyde (0.11 mL, 1.1 mmol), CH₂Cl₂ (10 mL), 2-chloropyridine (0.11 mL, 1.2 mmol), and Tf₂O (0.19 mL, 1.1 mmol). The reaction proceeded for 24 h at rt after addition of Tf₂O and was worked up as described above. The residue was purified by MPLC (47-68% EtOAc in hexanes as eluent on an amino functionalized silica gel column) to afford the desired product (0.116 g, 38% yield) as a solid (m.p.=168-170° C.). ¹H NMR (400 MHz, CDCl₃) δ 7.33-7.16 (m, 5H), 6.70 (d, J=2.6 Hz, 1H), 6.61 (dd, J=8.3, 0.6 Hz, 1H), 6.46 (dd, J=8.4, 2.6 Hz, 1H), 5.42 (s, 1H), 3.75 (s, 3H), 3.39 (ddd, J=15.1, 8.9, 1.3 Hz, 1H), 3.19 (ddd, J=15.3, 8.5, 1.5 Hz, 1H), 2.73-2.56 (m, 2H), 1.81-1.68 (m, 2H), 1.68-1.42 (m, 3H), 1.30-1.16 (m, 1H); ¹³C NMR (101 MHz, CDCl₃) δ 161.9, 159.6, 145.1, 142.6, 128.9, 127.9, 127.1, 126.9, 117.5, 111.4, 107.7, 66.2, 55.2, 52.2, 37.4, 29.8, 28.3, 25.1; IR (neat): 2928, 1586, 1552, 1497, 1441, 1202, 1142 cm⁻¹; HRMS (ESI): m/z calcd for C₂₀H₂₃N₂O [M+H], 307.1810; found, 307.1811.

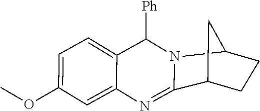 [SMILES: COc1ccc2c(c1)N=C1C3CCC(C3)N1C2c1ccccc1]

Prepared according to the general dihydroquinazoline protocol with cis-3-amino-N-(3-methoxyphenyl)cyclopentanecarboxamide (0.118 g, 0.50 mmol), benzaldehyde (0.060 mL, 0.55 mmol), CH₂Cl₂ (10 mL), 2-chloropyridine (0.060 mL, 0.60 mmol), and Tf₂O (0.090 mL, 0.55 mmol). The reaction proceeded for 24 h at rt after addition of Tf₂O and was worked up as described above. The residue was purified by MPLC (50-100% EtOAc in hexanes as eluent on an amino functionalized silica gel column) to afford the desired product (0.059 g, 39% yield) as a solid (m.p.=160-164° C.). ¹H NMR (400 MHz, CDCl₃) δ 7.45-7.30 (m, 5H), 6.69 (d, J=2.5 Hz, 1H), 6.42 (dd, J=8.5, 2.6 Hz, 1H), 6.38 (dd, J=8.4, 0.9 Hz, 1H), 5.47 (s, 1H), 3.75 (s, 3H), 3.44 (dd, J=2.3, 1.2 Hz, 1H), 3.11 (dd, J=3.1, 1.4 Hz, 1H), 1.99-1.81 (m, 2H), 1.79-1.60 (m, 3H), 1.32 (dt, J=9.3, 1.4 Hz, 1H); ¹³C NMR (101 MHz, CDCl₃) δ 166.4, 159.8, 144.0, 142.2, 128.9, 128.8, 128.2, 128.1, 117.4, 110.8, 108.4, 59.1, 58.1, 55.3, 45.1, 39.0, 26.1, 24.7; IR (neat): 2954, 1636, 1601, 1491, 1273, 1154 cm⁻¹; HRMS (ESI): m/z calcd for C₂₀H₂₁N₂O [M+H], 305.1654; found, 305.1652.

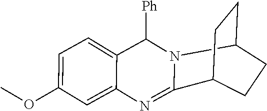 [SMILES: COc1ccc2c(c1)N=C1C3CCC(CC3)N1C2c1ccccc1]

Prepared according to the general dihydroquinazoline protocol with cis-4-amino-N-(3-methoxyphenyl)cyclohexanecarboxamide (0.125 g, 0.50 mmol), benzaldehyde (0.060 mL, 0.55 mmol), CH₂Cl₂ (25 mL), 2-chloropyridine (0.060 mL, 0.60 mmol), and Tf₂O (0.090 mL, 0.55 mmol). The reaction proceeded for 24 h at rt after addition of Tf₂O and was worked up as described above. The residue was purified by MPLC (50-100% EtOAc in hexanes as eluent on an amino functionalized silica gel column) to afford the desired product (0.042 g, 26% yield) as a solid (m.p.=126-130° C.). ¹H NMR (400 MHz, CDCl₃) δ 7.44-7.33 (m, 5H), 6.88 (d, J=2.3 Hz, 1H), 6.53-6.40 (m, 2H), 5.68 (s, 1H), 3.78 (s, 3H), 3.38 (t, J=4.8 Hz, 1H), 3.13 (s, 1H), 2.06 (tdd, J=7.9, 5.3, 2.4 Hz, 2H), 1.93 (d, J=13.9 Hz, 1H), 1.83-1.62 (m, 3H), 1.57 (d, J=10.9 Hz, 1H), 1.50-1.34 (m, 1H); ¹³C NMR (101 MHz, CDCl₃) δ 164.4, 159.9, 141.6, 140.7, 129.0, 128.5, 128.4, 128.3, 115.6, 111.9, 106.8, 58.4, 56.7, 55.4, 40.7, 37.1, 27.5, 23.1, 18.3; IR (neat): 2939, 1653, 1604, 1485, 1286, 1154 cm¹; HRMS (ESI): m/z calcd for C₂₁H₂₃N₂O [M+H], 319.1810; found, 319.1813.

1. A compound of the formula (I): 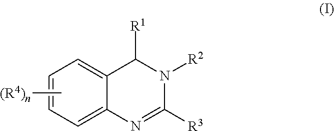
or a pharmaceutically acceptable salt, polymorph, prodrug, solvate or clathrate thereof, wherein:
R¹ is aryl or heteroaryl;
R² is alkyl substituted with cyano; alkoxy; alkoxyalkyl; aryl; (C₄-C₁₀)alkenyl; cycloalkylalkyl; alkynyl; arylalkyl; substituted heteroaryl; (C₂-C₄)heteroaryl or heterocyclylalkyl;
R³ is aryl, cycloalkyl, (C₅-C₁₀)alkyl;
each R⁴ is, independently, alkenyl, alkynyl, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein); and
n is an integer from 1 to 3.

2. The compound of claim 1, wherein the compound of formula (I) is a compound of the formula (Ia): 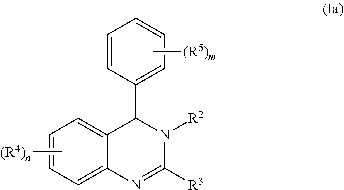
or a pharmaceutically acceptable salt, polymorph, prodrug, solvate or clathrate thereof, wherein:
R² is alkyl substituted with cyano; alkoxy; alkoxyalkyl; aryl; (C₄-C₁₀)alkenyl; cycloalkylalkyl; alkynyl; arylalkyl; substituted heteroaryl; (C₂-C₄)heteroaryl or heterocyclylalkyl;
R³ is aryl, cycloalkyl, (C₅-C₁₀)alkyl;
each R⁴ is, independently, alkenyl, alkynyl, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
m is an integer from 1 to 3; and
n is an integer from 1 to 3.

3. The compound of claim 1, wherein the compound of formula (I) is a compound of the formula (Ib): 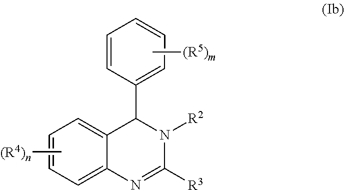
or a pharmaceutically acceptable salt, polymorph, prodrug, solvate or clathrate thereof, wherein:
R² is alkyl substituted with cyano; alkoxy; alkoxyalkyl; aryl; (C₄-C₁₀)alkenyl; cycloalkylalkyl; alkynyl; arylalkyl; substituted heteroaryl; (C₂-C₄)heteroaryl or heterocyclylalkyl;
R³ is aryl, cycloalkyl, (C₅-C₁₀)alkyl;
each R⁴ is, independently, alkenyl, alkynyl, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
m is an integer from 1 to 3; and
n is an integer from 1 to 3.

4. A compound of the formula (Ic): 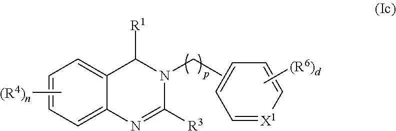
or a pharmaceutically acceptable salt, polymorph, prodrug, solvate or clathrate thereof, wherein:
X¹ is N or CH;
R¹ is aryl or heteroaryl;
R³ is aryl, cycloalkyl, (C₅-C₁₀)alkyl
each R⁴ is, independently, alkenyl, alkynyl, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein); and
each R⁶ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein) or two R⁶ groups on adjacent carbon atoms, together with the carbon atoms to which they are attached, form an aryl or heterocyclyl group;
n and d are each, independently, an integer from 1 to 3; and
p is an integer from 0 to 10, with the proviso that p is not 0 when X¹ is N and R⁶ is H.

5. A compound of the formula (Id): 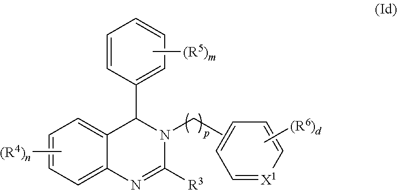
or a pharmaceutically acceptable salt, polymorph, prodrug, solvate or clathrate thereof, wherein:
X¹ is N or CH;
R³ is aryl, cycloalkyl, (C₅-C₁₀)alkyl
each R⁴ is, independently, alkenyl, alkynyl, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁶ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein) or two R⁶ groups on adjacent carbon atoms, together with the carbon atoms to which they are attached, form an aryl or heterocyclyl group;
m, n, and d are each, independently, an integer from 1 to 3; and
p is an integer from 0 to 10, with the proviso that p is not 0 when X¹ is N and R⁶ is H.

6. A compound of the formula (Ie): 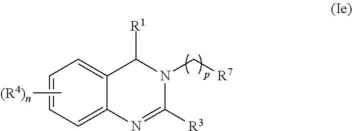
or a pharmaceutically acceptable salt, polymorph, prodrug, solvate or clathrate thereof, wherein:
R¹ is aryl or heteroaryl;
R³ is aryl, cycloalkyl, (C₅-C₁₀)alkyl;
each R⁴ is, independently, H, alkyl, alkenyl, alkynyl, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
R¹ is cycloalkyl or heterocyclylalkyl;
n is an integer from 1 to 3; and
p is an integer from 0 to 10.

7. A compound of the formula (If): 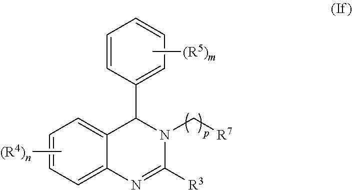
or a pharmaceutically acceptable salt, polymorph, prodrug, solvate or clathrate thereof, wherein:
R³ is aryl, cycloalkyl, (C₅-C₁₀)alkyl;
each R⁴ is, independently, H, alkyl, alkenyl, alkynyl, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
R⁷ is cycloalkyl or heterocyclylalkyl;
m and n are each, independently, an integer from 1 to 3; and
p is an integer from 0 to 10, with the proviso that R⁷ is not: 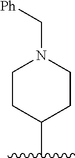 [SMILES: *C1CCN(Cc2ccccc2)CC1]
when p is 0.

8. A compound of the formula (Ig): 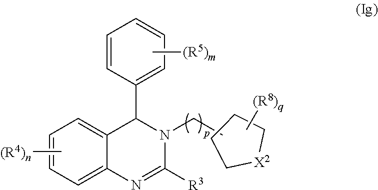
or a pharmaceutically acceptable salt, polymorph, prodrug, solvate or clathrate thereof, wherein:
X² is absent, CH₂, CH₂CH₂, O, OCH₂, NR⁹, wherein R⁹ is H or alkyl, or CH₂NR⁹;
R³ is aryl, cycloalkyl, (C₅-C₁₀)alkyl;
each R⁴ is, independently, H, alkyl, alkenyl, alkynyl, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁸ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
m and n are each, independently, an integer from 1 to 3;
q is an integer from 1 to 2; and
p is an integer from 0 to 10.

9. A compound of the formula (Ih): 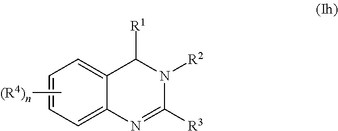
or a pharmaceutically acceptable salt, polymorph, prodrug, solvate or clathrate thereof, wherein:
R¹ is aryl or heteroaryl;
R² and R³, together with the atoms to which they are attached, form a heterocyclic ring;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein); and
n is an integer from 1 to 3.

10. The compound of claim 9, wherein the heterocyclic ring is a C₄-C₁₀-heterocyclic ring.

11. The compound of claim 9, wherein the heterocyclic ring is a saturated C₄-C₁₀-heterocyclic ring.

12. The compound of claim 9, wherein the compound of formula (Ih) is:

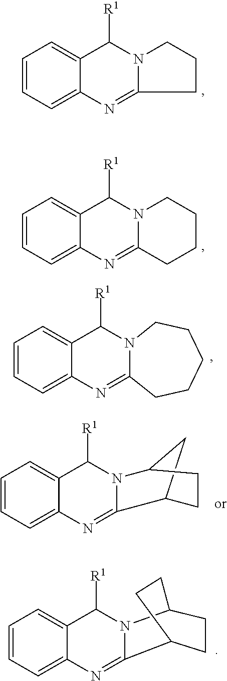

13. A pharmaceutical composition comprising one or more compounds any preceding claim and one or more pharmaceutically acceptable excipients.

14. A method for reducing, substantially eliminating or eliminating dysregulation of proteostasis comprising administering a therapeutically effective amount of one or more compounds of the formula (I): 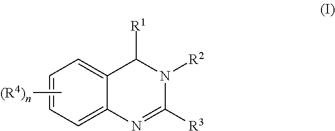
or a pharmaceutically acceptable salt, polymorph, prodrug, solvate or clathrate thereof, wherein:
R¹ is aryl or heteroaryl;
R² is alkyl substituted with cyano; alkoxy; alkoxyalkyl; aryl; (C₄-C₁₀)alkenyl; cycloalkylalkyl; alkynyl; arylalkyl; substituted heteroaryl; (C₂-C₄)heteroaryl or heterocyclylalkyl;
R³ is aryl, cycloalkyl, (C₅-C₁₀)alkyl;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein); and
n is an integer from 1 to 3.

15. The method of claim 14, wherein the compound of formula (I) is a compound of the formula (Ia): 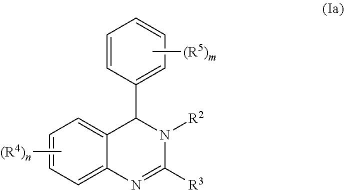
or a pharmaceutically acceptable salt, polymorph, prodrug, solvate or clathrate thereof, wherein:
R² is alkyl substituted with cyano; alkoxy; alkoxyalkyl; aryl; (C₄-C₁₀)alkenyl; cycloalkylalkyl; alkynyl; arylalkyl; substituted heteroaryl; (C₂-C₄)heteroaryl or heterocyclylalkyl;
R³ is aryl, cycloalkyl, (C₅-C₁₀)alkyl;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
m is an integer from 1 to 3; and
n is an integer from 1 to 3.

16. The method of claim 14, wherein the compound of formula (I) is a compound of the formula (Ib): 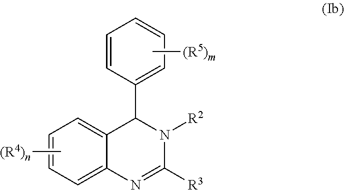
or a pharmaceutically acceptable salt, polymorph, prodrug, solvate or clathrate thereof, wherein:
R² is alkyl substituted with cyano; alkoxy; alkoxyalkyl; aryl; (C₄-C₁₀)alkenyl; cycloalkylalkyl; alkynyl; arylalkyl; substituted heteroaryl; (C₂-C₄)heteroaryl or heterocyclylalkyl;
R³ is aryl, cycloalkyl, (C₅-C₁₀)alkyl;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
m is an integer from 1 to 3; and
n is an integer from 1 to 3.

17. The method of claim 14, wherein the compound of formula (I) is a compound of the formula (Ic): 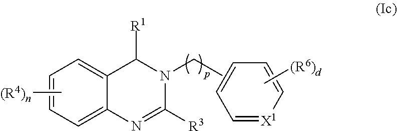
or a pharmaceutically acceptable salt, polymorph, prodrug, solvate or clathrate thereof, wherein:
X¹ is N or CH;
R¹ is aryl or heteroaryl;
R³ is aryl, cycloalkyl, (C₅-C₁₀)alkyl
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein); and
each R⁶ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein) or two R⁶ groups on adjacent carbon atoms, together with the carbon atoms to which they are attached, form an aryl or heterocyclyl group;
n and d are each, independently, an integer from 1 to 3; and
p is an integer from 0 to 10, with the proviso that p is not 0 when X¹ is N and R⁶ is H.

18. The method of claim 14, wherein the compound of formula (I) is a compound of the formula (Id): 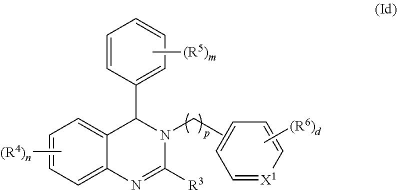
or a pharmaceutically acceptable salt, polymorph, prodrug, solvate or clathrate thereof, wherein:
X¹ is N or CH;
R³ is aryl, cycloalkyl, (C₅-C₁₀)alkyl
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁶ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein) or two R⁶ groups on adjacent carbon atoms, together with the carbon atoms to which they are attached, form an aryl or heterocyclyl group;
m, n, and d are each, independently, an integer from 1 to 3; and
p is an integer from 0 to 10, with the proviso that p is not 0 when X¹ is N and R⁶ is H.

19. The method of claim 14, wherein the compound of formula (I) is a compound of the formula (Ie): 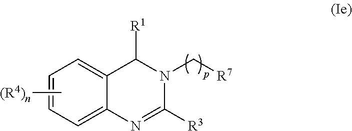
or a pharmaceutically acceptable salt, polymorph, prodrug, solvate or clathrate thereof, wherein:
R¹ is aryl or heteroaryl;
R³ is aryl, cycloalkyl, (C₅-C₁₀)alkyl;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
R¹ is cycloalkyl or heterocyclylalkyl;
n is an integer from 1 to 3; and
p is an integer from 0 to 10.

20. The method of claim 14, wherein the compound of formula (I) is a compound of the formula (If): 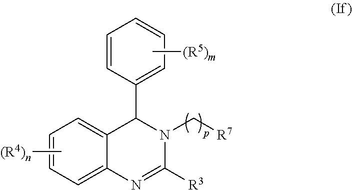
or a pharmaceutically acceptable salt, polymorph, prodrug, solvate or clathrate thereof, wherein:
R³ is aryl, cycloalkyl, (C₅-C₁₀)alkyl;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
R¹ is cycloalkyl or heterocyclylalkyl;
m and n are each, independently, an integer from 1 to 3; and
p is an integer from 0 to 10, with the proviso that R¹ is not: 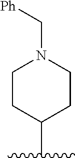 [SMILES: *C1CCN(Cc2ccccc2)CC1]
when p is 0.

21. The method of claim 14, wherein the compound of formula (I) is a compound of the formula (Ig): 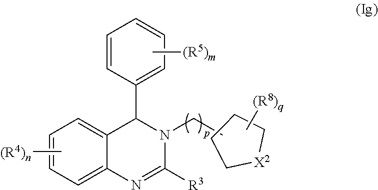
or a pharmaceutically acceptable salt, polymorph, prodrug, solvate or clathrate thereof, wherein:
X² is absent, CH₂, CH₂CH₂, O, OCH₂, NR⁹, wherein R⁹ is H or alkyl, or CH₂NR;
R³ is aryl, cycloalkyl, (C₅-C₁₀)alkyl;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R⁵ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
each R¹ is, independently, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein);
m and n are each, independently, an integer from 1 to 3;
q is an integer from 1 to 2; and
p is an integer from 0 to 10.

22. The method of claim 14, wherein the compound is a compound of the formula: 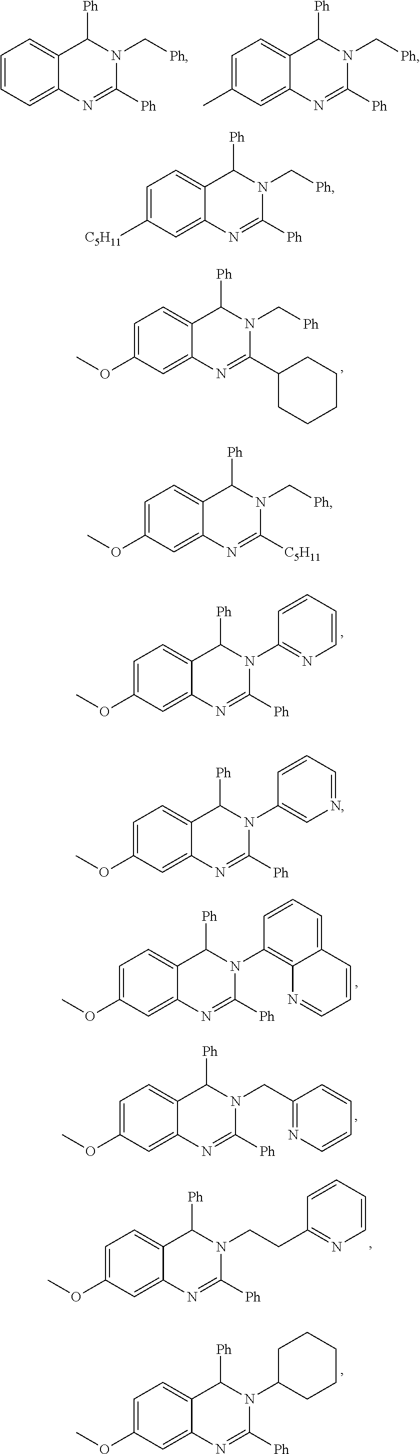 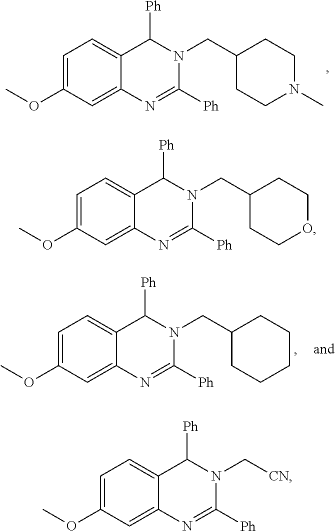
or pharmaceutically acceptable salts, polymorphs, prodrugs, solvates or clathrates thereof.

23. A method for reducing, substantially eliminating or eliminating dysregulation of proteostasis comprising administering a therapeutically effective amount of one or more compounds of the formula (Ih): 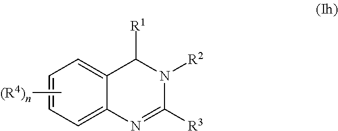
or a pharmaceutically acceptable salt, polymorph, prodrug, solvate or clathrate thereof, wherein:
R¹ is aryl or heteroaryl;
R² and R³, together with the atoms to which they are attached, form a heterocyclic ring;
each R⁴ is, independently, H, alkyl, alkenyl, alkynyl, alkoxy, halo, cycloalkyl, cycloalkenyl, aryl, heterocyclyl, cyano, amino, acyl, carboxylate and esters thereof, amido, S(O)ₓR (wherein x is 0, 1 or 2 and R is defined herein), SO₂N(R)₂ (wherein R is defined herein), P(O)(OR)₂ (wherein R is defined herein) or OP(O)(OR)₂ (wherein R is defined herein); and
n is an integer from 1 to 3.
